(19) United States
(12) Patent Application Publication
(10) Pub. No.: US 2017/0168124 A1
(43) Pub. Date: Jun. 15, 2017

(54) MEDICAL IMAGE DIAGNOSTIC APPARATUS

(71) Applicant: Toshiba Medical Systems Corporation, Otawara-shi (JP)

(72) Inventors: Yu UEDA, Utsunomiya (JP); Hiromitsu TAKAMORI, Yokohama (JP)

(73) Assignee: Toshiba Medical Systems Corporation, Otawara-shi (JP)

(21) Appl. No.: 15/373,092

(22) Filed: Dec. 8, 2016

(30) Foreign Application Priority Data
Dec. 11, 2015 (JP) 2015-242244

(51) Int. Cl. G01R 33/28; H05B 37/02; G02B 6/32; G02B 6/42; A61B 5/055; G02B 27/14; F21V 8/00; G03B 21/56; A61B 5/00; H04N 9/31; G02B 7/182

(52) U.S. Cl. CPC G01R 33/283; H04N 9/3155; H04N 9/3185; H04N 9/312; H04N 9/3105; H04N 9/3194; H05B 37/0209; G02B 6/32; G02B 6/42; G02B 7/182; G02B 27/14; G02B 6/005; G03B 21/562; A61B 5/0035; A61B 5/0077; A61B 5/0555; A61B 5/742

(57) ABSTRACT

According to one embodiment, a medical image diagnostic apparatus includes an image providing apparatus and control circuitry. The image providing apparatus provides an image from an image source for a subject placed on a couch top via a reflecting plate which reflects the image or a display which displays the image. The control circuitry performs at least one of control of illumination provided in an examination room or a gantry and control of a camera which photographs the subject placed on the couch top, according to attachment and detachment of the image providing apparatus or positioning of the image providing apparatus with respect to the couch top.

### CROSS-REFERENCE TO RELATED APPLICATIONS

This application is based upon and claims the benefit of priority from the prior Japanese Patent Application No. 2015-242244, filed Dec. 11, 2015, the entire contents of which are incorporated herein by reference.

### FIELD

Embodiments described herein relate generally to a medical image diagnostic apparatus.

### BACKGROUND

A magnetic resonance diagnostic apparatus has a gantry which is equipped with an imaging mechanism such as a magnet. A bore having a substantially hollow shape is formed in the gantry. MR (magnetic resonance) imaging is performed in a state in which a patient is inserted inside a bore. A gantry having a relatively large bore diameter has been developed, but not a few patients feel stress against MR examination due to MR imaging time requires a long period of time, noise during driving of the gantry, and a sense of pressure and a sense of blockage inside a bore.

### BRIEF DESCRIPTION OF THE DRAWINGS

FIG. 1 is a diagram illustrating a configuration of a medical image diagnostic system that includes a medical image diagnostic apparatus according to an embodiment of the present invention.

FIG. 2 is a diagram illustrating a configuration of a magnetic resonance diagnostic apparatus according to the present embodiment.

FIG. 3 is a diagram illustrating an example of installation environment of a magnetic resonance imaging system according to the present embodiment.

FIG. 4 is a perspective view of a gantry housing according to the present embodiment.

FIG. 5 is a perspective view of a movable screen apparatus according to the present embodiment.

FIG. 6 relates to the present embodiment and is a side view of the movable screen apparatus of FIG. 5.

FIG. 7 relates to the present embodiment and is a front view of the movable screen apparatus of FIG. 5.

FIG. 8 is a perspective view of the movable screen apparatus and a couch top, which are coupled with each other, according to the present embodiment.

FIG. 9 is a schematic front view of a screen which is arranged inside a bore according to the present embodiment.

FIG. 10 is a cross-sectional view illustrating an example of a configuration of a reflection unit according to the present embodiment.

FIG. 11 relates to the present embodiment and is a cross-sectional view illustrating an example of a reflection unit having a different configuration from that of FIG. 10.

FIG. 12 relates to the present embodiment and is a cross-sectional view illustrating an example of a reflection unit having a different configuration from those of FIGS. 10 and 11.

FIG. 13 relates to the present embodiment and is a side view of the movable screen apparatus whose support arm illustrated in FIG. 6 is slid in relation to a Z-axis.

FIG. 14 is a simple side view of the movable screen apparatus which is arranged in the bore of a gantry according to the present embodiment.

FIG. 15 is a diagram illustrating the movable screen apparatus in a first projection format according to the present embodiment from a lateral side of the gantry.

FIG. 16 is a diagram illustrating the movable screen apparatus in the first projection format according to the present embodiment from a front face of the gantry.

FIG. 17 is a diagram illustrating the movable screen apparatus in a second projection format according to the present embodiment from the lateral side of the gantry.

FIG. 18 relates to the present embodiment and is a diagram illustrating an example of attachment or detachment of a reflecting plate to or from the support arm.

FIG. 19 relates to the present embodiment and is a diagram illustrating an example of a rotating operation of the reflecting plate about a rotation shaft RR1.

FIG. 20 relates to the present embodiment and is a diagram illustrating an example of movement (slide) of the support arm with respect to the screen, the present embodiment.

FIG. 21 relates to the present embodiment and is a diagram illustrating a patient placed on the couch top before being inserted into the bore and the movable screen apparatus arranged at an end of the bore on a couch side from the lateral side of the gantry.

FIG. 22 relates to the present embodiment and is a diagram schematically illustrating the reflection unit arranged at a first angle in FIG. 21 and an eye of the patient.

FIG. 23 relates to the present embodiment and is a diagram illustrating an example of an image of a film which is arranged at the back of a beam splitter in FIGS. 21 and 22.

FIG. 24 relates to the present embodiment and is a diagram schematically illustrating the reflection unit arranged at a second angle and the patient's eye.

FIG. 25 relates to the present embodiment and is a diagram illustrating an example of the image of the film which is arranged at the back of the beam splitter in FIG. 24.

FIG. 26 relates to a second application example of the present embodiment and is a diagram illustrating an example of the movable screen apparatus which includes the reflection unit arranged at the first angle in a first positioning mode.

FIG. 27 relates to the second application example of the present embodiment and is a diagram illustrating an example of the movable screen apparatus at a time at which the support arm is slid to the couch side.

FIG. 28 relates to the second application example of the present embodiment and is a diagram illustrating an example of the movable screen apparatus in a state in which supply of current to a light source is cut off.

FIG. 29 is a flowchart illustrating an example of procedure of a process relating to a reflection state changing function according to the second application example of the present embodiment.

FIG. 30 relates to a third application example of the present embodiment and is a side view illustrating an example of a side face of the movable screen apparatus which includes the reflecting plate arranged at the first angle.

FIG. 31 relates to the third application example of the present embodiment and is a side view illustrating an example of the side face of the movable screen apparatus which includes the reflecting plate arranged at the second angle.

FIG. 32 relates to the third application example of the present embodiment and is a diagram illustrating an example of the reflecting plate arranged at the first angle in which a first objective lens is provided in the support arm.

FIG. 33 relates to the third application example of the present embodiment and is a diagram illustrating an example of the reflecting plate arranged at the second angle in which the first objective lens is provided in the support arm.

FIG. 34 relates to the third application example of the present embodiment and is a diagram illustrating a field of view of a first camera and a field of view of a second camera in a case in which the first camera is placed on the support arm and the reflecting plate is arranged at the second angle.

FIG. 35 relates to the third application example of the present embodiment and is a front view of a touch panel on which a facial image of the patient in FIG. 21 is displayed when viewed from the gantry on the couch side of.

FIG. 36 relates to a fourth application example of the present embodiment and is a side view illustrating an example of a side face of a movable screen apparatus 15 which includes a reflecting plate arranged at the first angle.

FIG. 37 relates to the fourth application example of the present embodiment and is a side view of the movable screen apparatus 15 which includes the reflecting plate arranged at the second angle.

FIG. 38 relates to a fifth application example of the present embodiment and is a diagram illustrating an example of each illumination state inside an examination room and the bore when the reflecting plate is arranged at the first angle as a positioning state in the first positioning mode.

FIG. 39 relates to the fifth application example of the present embodiment and is a diagram illustrating an example of each illumination state inside the examination room and a bore 53 when the reflecting plate is arranged at the second angle as a positioning state.

FIG. 40 relates to a sixth application example of the present embodiment and is a diagram illustrating an example of an illumination state of a light emitter when the reflecting plate is arranged at the first angle as a positioning state in the first positioning mode.

FIG. 41 is a side view of the movable screen apparatus according to a seventh application example of the present embodiment.

### DETAILED DESCRIPTION

In general, according to one embodiment, a medical image diagnostic apparatus includes an image providing apparatus and control circuitry. The image providing apparatus provides an image from an image source for a subject placed on a couch top via a reflecting plate which reflects the image or a display which displays the image. The control circuitry performs at least one of control of illumination provided in an examination room or a gantry and control of a camera which photographs the subject placed on the couch top, according to attachment and detachment of the image providing apparatus or positioning of the image providing apparatus with respect to the couch top.

The following technique is considered as a technique to mitigate stress during MR examination. Examples of the technique include: 1. a goggle-type head-mounted display; 2. installation of a liquid crystal monitor on a ceiling or wall of an examination room; 3. a head coil attached with a mirror to show an image on a liquid crystal monitor arranged at the rear side of a gantry; and 4. a movable body that has a mirror to show an image projected on a screen provided at the rear side of a patient's head and can be moved inside a bore. However, a patient feels a sense of pressure and a sense of blockage as the head-mounted display is attached to the patient in the case of Technique 1. It is difficult to view an image on the liquid crystal monitor if a patient's head enters a gantry in the case of Technique 2.

It is possible to view the image through the mirror mounted to the head coil during MR imaging, and thus, it is possible to mitigate the sense of blockage inside the bore in the case of Technique 3. However, it is necessary to attach the mirror to each head coil. In addition, the mirror is attached to a gap between the head coils covering the head, and thus, the patient hardly feels an extent of the image. In addition, the liquid crystal monitor is installed at the rear side of the gantry and does not hide the front of the gantry, and thus, the patient can visually recognize the bore easily when being outside the gantry before the MR imaging, and hardly dispels a sense of being inside the bore even if viewing the image through the mirror using a head coil after then. Further, a positional relationship between the mirror and the liquid crystal monitor is changed along with movement of a couch top, and thus, the patient still feels a sense of passing inside the bore even when viewing the image on the liquid crystal monitor through the mirror during the movement of the couch top.

The mirror in a horizontal state is drawn out in a visual angle range of the patient placed on a couch top in the case of Technique 4. At this time, the patient's own face is reflected on the mirror, and thus, the patient feels the sense of discomfort in some cases. In addition, it is necessary to move the patient's head to a lower side of a projector at the time of performing positioning in order to photograph the patient's head. Thus, the patient is difficult to visually recognize the image projected on a screen through the mirror in some cases. Accordingly, a sense of comfort inside examination space is impaired even in the case of Technique 4 in some cases.

Hereinafter, a description will be given regarding a medical image diagnostic apparatus and a magnetic resonance diagnostic apparatus according to the present embodiment with reference to the drawings.

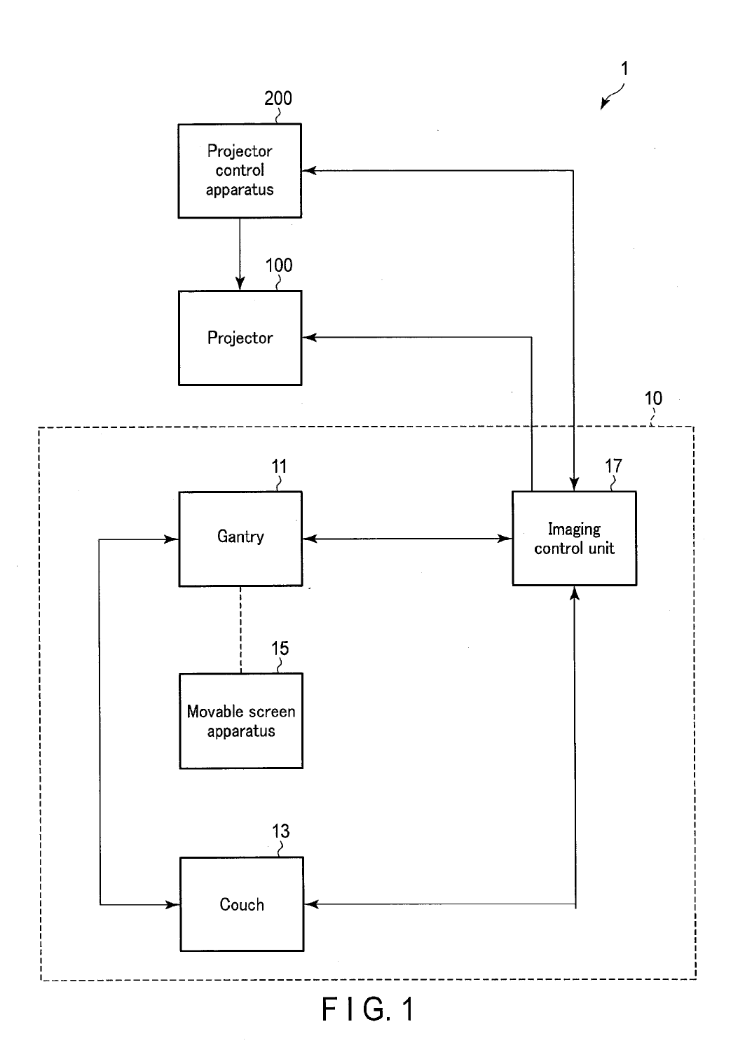

FIG. 1 is a diagram illustrating a configuration of a medical image diagnostic system 1 that includes a medical image diagnostic apparatus 10 according to the present embodiment. As illustrated in FIG. 1, the medical image diagnostic system 1 includes the medical image diagnostic apparatus 10, a projector 100, and a projector control apparatus 200 which are connected in a wired or wireless manner to be capable of communicating with each other. The medical image diagnostic apparatus 10 includes a gantry 11, a couch 13, a movable screen apparatus (video projection apparatus) 15, and an imaging control unit 17. For example, the gantry 11, the couch 13, and the movable screen apparatus 15 are installed in the examination room, and the imaging control unit 17 is installed in a control room adjacent to the examination room.

The gantry 11 is equipped with a mechanism configured to realize medical imaging. A bore having a hollow shape is formed in the gantry 11. The couch 13 is installed at the front side of the gantry 11. The couch 13 supports a couch top on which a subject (hereinafter, a patient P, for example) is placed in a freely movable manner. The couch 13 moves the couch top according to control through the gantry 11, a console and the like. The movable screen apparatus 15 is movably provided in the bore of the gantry 11. The projector 100 is installed at the front side or the rear side of the gantry 11. An image from the projector 100 is projected on the movable screen apparatus 15.

The projector control apparatus 200 is a computer apparatus that controls the projector 100. The projector control apparatus 200 supplies data relating to the image as a projection target to the projector 100. The projector 100 projects the image corresponding to the data supplied from the projector control apparatus 200 on a screen of the movable screen apparatus 15. For example, a liquid crystal system, a digital light processing (DLP) system, a liquid crystal on silicon (LCOS) system, a grating light valve (GLV) system, or the like is preferably used as the projector 100.

In this case, at least a display device and a light source are mounted to the projector 100. The display device displays the image corresponding to the data supplied from the projector control apparatus 200. The light source irradiates the display device with light directly or indirectly via an optical system. The light (hereinafter, referred to as projected light) passing through or reflected by the display device is emitted to the outside of the projector 100 directly or indirectly via the optical system. When the movable screen apparatus 15 is irradiated with the projected light, the image corresponding to the projected light is projected on the movable screen apparatus 15.

The imaging control unit 17 functions as a hub of the medical image diagnostic apparatus 10. For example, the imaging control unit 17 controls the gantry 11 in order to perform the medical imaging. In addition, the imaging control unit 17 reconstructs a medical image relating to the patient P based on raw data collected by the gantry 11 in the medical imaging. Incidentally, the imaging control unit 17 may be configured to be capable of controlling the projector 100 via the projector control apparatus 200. In addition, the imaging control unit 17 may supply data relating to an image as a projection target to the projector 100. In this case, the projector 100 projects the image corresponding to the data supplied from the imaging control unit 17 on the screen of the movable screen apparatus 15.

Incidentally, the configuration of the medical image diagnostic system 1 according to the present embodiment is not limited only to the above-described configuration. For example, the projector control apparatus 200 is not necessarily provided in the medical image diagnostic system 1 as long as the imaging control unit 17 has the above-described function of controlling the projector 100 according to the projector control apparatus 200.

The medical image diagnostic system 1 according to the present embodiment can enhance dwelling ability inside the bore during the medical imaging according to the medical image diagnostic apparatus 10 by utilizing the projector 100 and the movable screen apparatus 15. An apparatus that can image the patient P using the gantry 11 in which the bore is formed may be used as the medical image diagnostic apparatus 10 according to the present embodiment. To be specific, a single modality such as a magnetic resonance imaging (MRI) apparatus, an X-ray computed tomography (CT) apparatus, a positron emission tomography (PET) apparatus, and a single photon emission computed tomography (SPECT) apparatus can be applied as the medical image diagnostic apparatus 10 according to the present embodiment.

Alternatively, a combined modality such as an MR/PET apparatus, a CT/PET apparatus, an MR/SPECT apparatus, and a CT/SPECT apparatus may be applied as the medical image diagnostic apparatus 10 according to the present embodiment. However, the medical image diagnostic apparatus 10 according to the present embodiment is assumed to be a magnetic resonance diagnostic apparatus 10 in order to give the following description in detail. In addition, the medical image diagnostic system 1, which includes the magnetic resonance diagnostic apparatus 10, the projector 100, and the projector control apparatus 200, will be referred to as a magnetic resonance imaging system 1.

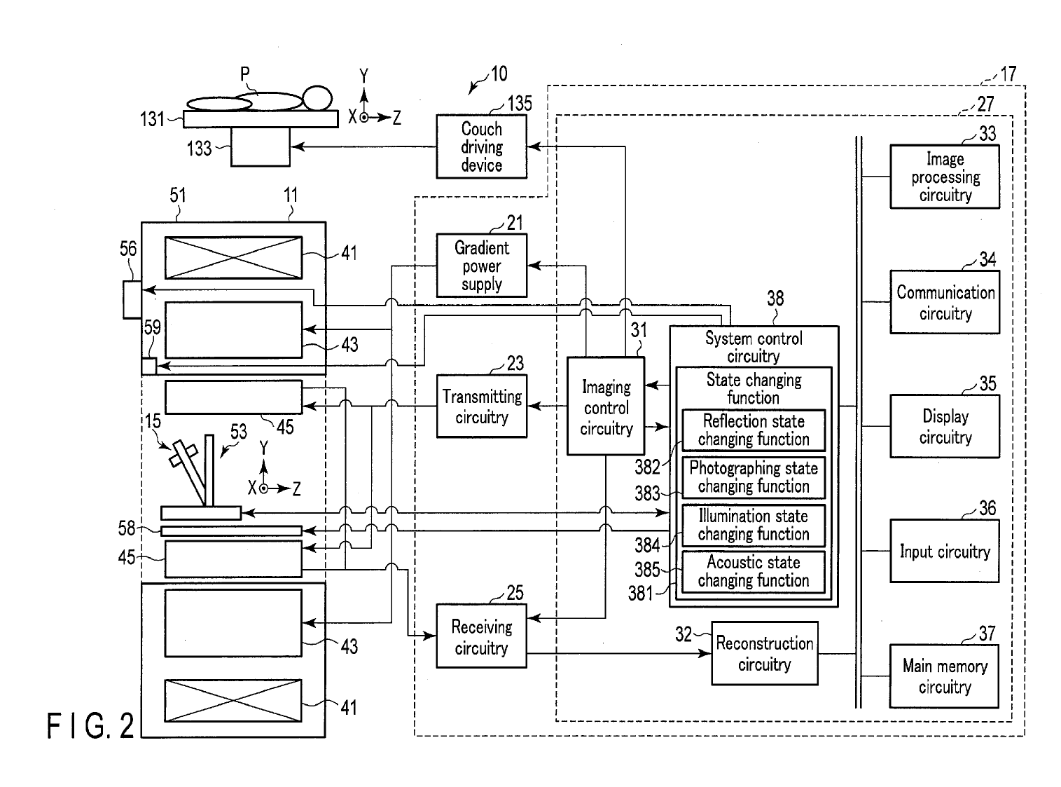

FIG. 2 is a diagram illustrating a configuration of the magnetic resonance diagnostic apparatus 10 according to the present embodiment. As illustrated in FIG. 2, the magnetic resonance diagnostic apparatus 10 includes the imaging control unit 17, the gantry 11, the couch 13, and the movable screen apparatus 15. The imaging control unit 17 includes a gradient power supply 21, transmitting circuitry 23, a receiving circuitry 25, and a console 27. The console 27 includes an imaging control circuitry 31, a reconstruction circuitry 32, an image processing circuitry 33, a communication circuitry 34, a display circuitry 35, an input circuitry 36, a main memory circuitry 37, and a system control circuitry 38.

The imaging control circuitry 31, the reconstruction circuitry 32, the image processing circuitry 33, the communication circuitry 34, the display circuitry 35, the input circuitry 36, the main memory circuitry 37, and the system control circuitry 38 are connected to be capable of communicating with each other via a bus. The gradient power supply 21, the transmitting circuitry 23, and the receiving circuitry 25 are provided separately from the console 27 and the gantry 11.

The gantry 11 includes a static field magnet 41, a gradient coil 43, an RF coil 45, an exterior illuminator 56, an in-bore illuminator 58, and a projector 59. In addition, the static field magnet 41 and the gradient coil 43 are housed in a housing (hereinafter, referred to as a gantry housing) 51 of the gantry 11. A bore 53 having a hollow shape is formed in the gantry housing 51. The RF coil 45 is arranged inside the bore 53 of the gantry housing 51. In addition, the movable screen apparatus 15 according to the present embodiment is arranged inside the bore 53 of the gantry housing 51. The exterior illuminator 56, the in-bore illuminator 58, and the projector 59 will be described later.

The static field magnet 41 has a substantially cylindrical shape with a hollow, and generates a static magnetic field in the substantially cylindrical interior. For example, a permanent magnet, a superconducting magnet, a normal conducting magnet, or the like is used as the static field magnet 41. Herein, a central axis of the static field magnet 41 is defined as a Z-axis, and an axis which is vertically orthogonal to the Z-axis is referred to as a Y-axis, and an axis which is horizontally orthogonal to the Z-axis is referred to as an X-axis. The X-axis, the Y-axis, and the Z-axis form a three-dimensional coordinate system orthogonal to each other.

The gradient coil 43 is a coil unit which is attached to an inner side of the static field magnet 41 and is formed in a substantially cylindrical shape with a hollow. The gradient coil 43 receives supply of current from the gradient power supply 21 and generates a gradient magnetic field.

The gradient power supply 21 supplies the current to the gradient coil 43 according to control of the imaging control circuitry 31. The gradient power supply 21 causes the gradient coil 43 to generate the gradient magnetic field by supplying the current to the gradient coil 43.

The RF coil 45 is arranged at an inner side of the gradient coil 43 and generates a high frequency magnetic field by receiving supply of a RF pulse from the transmitting circuitry 23. In addition, the RF coil 45 receives a magnetic resonance signal (hereinafter, referred to as an MR signal), which is emitted from a target atomic nucleus present inside the patient P by receiving action of the high frequency magnetic field. The received MR signal is supplied to the receiving circuitry 25 in a wired or wireless manner. Incidentally, the above-described RF coil 45 is configured as a coil having the transmission and reception functions, but an RF coil for transmission and an RF coil for reception may be provided separately.

The transmitting circuitry 23 transmits the high frequency magnetic field for excitation of the target atomic nucleus present inside the patient P to the patient P via the RF coil 45. Typically, proton is used as the target atomic nucleus. To be specific, the transmitting circuitry 23 supplies a high frequency signal (RF signal) for excitation of the target atomic nucleus to the RF coil 45 according to control of the imaging control circuitry 31.

The high frequency magnetic field generated from the RF coil 45 oscillates with a unique resonant frequency with respect to the target atomic nucleus and excites the target atomic nucleus. The MR signal is generated from the excited target atomic nucleus, and is detected by the RF coil 45. The detected MR signal is supplied to the receiving circuitry 25.

The receiving circuitry 25 receives the MR signal, generated from the excited target atomic nucleus via the RF coil 45. The receiving circuitry 25 performs signal processing of the received MR signal to generate a digital MR signal. The digital MR signal is supplied to the reconstruction circuitry 32 in a wired or wireless manner.

The couch 13 is installed to be adjacent to the gantry 11. The couch 13 includes a couch top 131 and a base 133. The patient P is placed on the couch top 131. The base 133 supports the couch top 131 to be slidable along each of the X-axis, the Y-axis, and the Z-axis. A couch driving device 135 is housed in the base 133. The couch driving device 135 receives the control of the imaging control circuitry 31 and moves the couch top 131. Any motor such as a servomotor and a stepping motor may be used as the couch driving device 135.

The imaging control circuitry 31 includes a processor such as a central processing unit (CPU) or a micro processing unit (MPU) and a memory such as a read only memory (ROM) and a random access memory (RAM) as hardware resources. The imaging control circuitry 31 controls the gradient power supply 21, the transmitting circuitry 23, and the receiving circuitry 25 in a synchronous manner based on pulse sequence information supplied from the system control circuitry 38, and images the patient P at a pulse sequence according to the pulse sequence information.

The reconstruction circuitry 32 includes a processor such as a CPU, a graphical processing unit (GPU), and an MPU and a memory such as a ROM and a RAM as hardware resources. The reconstruction circuitry 32 reconstructs an MR image relating to the patient P based on the MR signal supplied from the receiving circuitry 25. For example, the reconstruction circuitry 32 generates the MR image which is defined in a real space by performing the Fourier transform or the like on the MR signal arranged in a k-space or a frequency space.

Incidentally, the reconstruction circuitry 32 may be realized by an application specific integrated circuit (ASIC), a field programmable logic array (FPGA), another complex programmable logic device (CPLD), a simple programmable logic device (SPLD) that realizes the reconstruction function.

The image processing circuitry 33 includes a processor such as a CPU, a GPU, and an MPU and a memory such as a ROM and a RAM as hardware resources. The image processing circuitry 33 performs various types of image processing with respect to the MR image reconstructed by the reconstruction circuitry 32. Incidentally the image processing circuitry 33 may be realized by an ASIC, an FPGA, a CPLD or an SPLD that realizes the above-described image processing function.

The communication circuitry 34 performs data communication with the projector control apparatus 200 or the projector 100 via a wire (not illustrated) or in a wireless manner. In addition, the communication circuitry 34 may perform data communication with an external apparatus such as a PACS server connected via network or the like (not illustrated). In addition, the communication circuitry 34 may perform data communication with a device to be described later which is attached to the movable screen apparatus 15.

The display circuitry 35 displays various types of information. For example, the display circuitry 35 displays the MR image reconstructed by the reconstruction circuitry 32 or the MR image after being subjected to the image processing by the image processing circuitry 33. In addition, the display circuitry 35 may display the image projected by the projector 100. To be specific, the display circuitry 35 includes a display interface circuitry and a display device. The display interface circuitry converts data representing a display target to a video signal. The display signal is supplied to the display device. The display device displays the video signal representing the display target.

For example, a CRT display, a liquid crystal display, an organic EL display, an LED display, a plasma display, or any arbitrary display which has been known in the technical field can be suitably used as the display device.

Incidentally, the display device may be additionally provided on an exterior surface of the gantry housing 51, for example, other than the console 27. At this time, an input device such as a pointing device having a pressure-sensitive system, an optical position detection system, or an electrostatic system is provided in front of the display device. For example, a touch panel (video display monitor: also referred to as a touch screen) in which the display device and the pointing device are integrated may be provided on the exterior surface of the gantry housing 51 on the couch 13 side.

To be specific, the input circuitry 36 includes an input device and an input interface circuitry. The input device receives various instructions from a user. A keyboard, a mouse, various switches, and the like can be used as the input device. The input interface circuitry supplies an output signal from the input device to the system control circuitry 38 via a bus. Incidentally, the input circuitry 36 is not limited to one provided with physical operating parts such as a mouse and a keyboard. Examples of the input circuitry 36 include a processing circuitry of an electrical signal that receives the electrical signal corresponding to an input operation from an external input device, provided separately from the magnetic resonance diagnostic apparatus 10, and outputs the received electrical signal to various circuits.

The main memory circuitry 37 is a storage apparatus that stores various types of information such as a hard disk drive (HDD), a solid state drive (SSD), and an integrated circuit storage apparatus. In addition, the main memory circuitry 37 may be a driving device that reads and writes various types of information with respect to a portable recording medium such as a CD-ROM drive, a DVD drive, and a flash memory. For example, the main memory circuitry 37 stores the MR image, a control program of the magnetic resonance diagnostic apparatus 10, and the like.

The system control circuitry 38 includes a processor such as a CPU and an MPU and a memory such as a ROM and a RAM as hardware resources. The system control circuitry 38 functions as a hub of the magnetic resonance diagnostic apparatus 10. To be specific, the system control circuitry 38 reads the control program stored in the main memory circuitry 37, develops the control program on the memory, and controls each unit of the magnetic resonance diagnostic apparatus 10 according to the developed control program.

The system control circuitry 38 controls each unit based on a detection result from a detector to be described later in order to change at least one state of an illumination state and an acoustic state of a space in which the movable screen apparatus 15 is arranged (the examination space inside the bore 53, the examination room, and the like), a photographing state to optically photograph the inside of the bore 53, and a reflection state of a reflection unit to be described later. At this time, the system control circuitry 38 functions as a state changing unit that realizes a state changing function 381 to be described later. The state changing function 381 will be described later.

Hereinafter, the magnetic resonance diagnostic apparatus 10 according to the present embodiment will be described in detail.

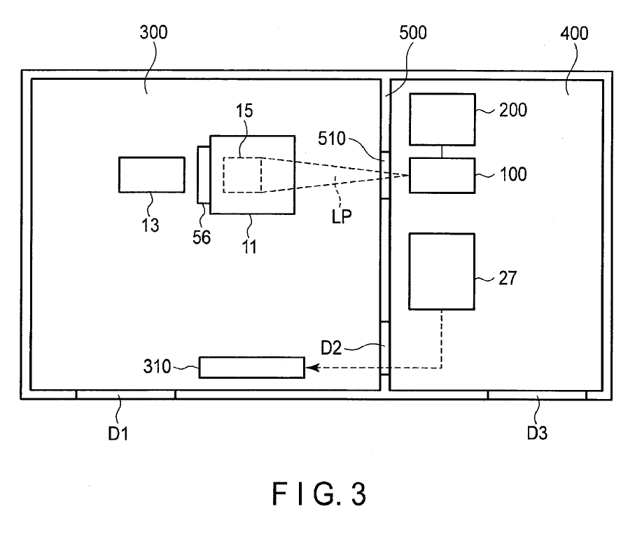

First, a description will be given regarding an installation environment of the magnetic resonance imaging system according to the embodiment 1 with reference to FIG. 3. FIG. 3 is a diagram illustrating an example of installation environment of a magnetic resonance imaging system according to the present embodiment. As illustrated in FIG. 3, an examination room 300 in which the MR imaging is performed and a control room 400 which is adjacent to the examination room 300 are provided. The gantry 11, the couch 13, and an examination room illuminator 310 are installed inside the examination room 300. The couch 13 is provided at the front side of the gantry 11. The movable screen apparatus 15 is provided in the bore of the gantry 11.

The examination room 300 is a shielded room that can shield a leaked magnetic field from the gantry 11, an electromagnetic field from the outside, and the like. A door D1, configured to allow entrance and exit, is provided in the examination room 300. In addition, a door D2, configured to allow coming and going between the examination room 300 and the control room 400, is provided between the examination room 300 and the control room 400. The console 27, the projector 100, and the projector control apparatus 200 are installed in the control room 400. The projector 100 is installed at the rear side of the gantry 11 being spaced apart from a wall 500 between the examination room 300 and the control room 400.

At least one examination room illuminator 310 is installed at an arbitrary position of the examination room 300 as illustrated in FIG. 3. The examination room illuminator 310 includes at least one light-emitting diode (LED). Incidentally, the examination room illuminator 310 may include a plurality of light-emitting diodes which generate light with a plurality of hues, respectively.

The examination room illuminator 310 generates light having an arbitrary hue and arbitrary brightness based on the control of the system control circuitry 38 or operation of a switch or the like (not illustrated) which is installed on a wall surrounding the examination room 300. Incidentally, an arbitrary light-emitting device (for example, a fluorescent tube and the like that has a non-magnetic or magnetically shielded property) may be used as a light source of the examination room illuminator 310 instead of the light-emitting diode.

Incidentally, the examination room illuminator 310 may include an optical element that diffuses and reflects the light generated by the light-emitting diode. At this time, the examination room illuminator 310 irradiates the inside of the examination room with the diffused light and the reflected light. The control of the examination room illuminator 310 performed by the system control circuitry 38 will be described later when describing an illumination state changing function 384.

A window 510 through which a projected light LP is transmitted is provided in a portion of the wall 500 in which a projected light LP from the projector 100 toward the movable screen apparatus 15 is propagated. It is possible to propagate the projected light LP from the projector 100 installed in the control room 400 to the movable screen apparatus 15 of the examination room 300 via the window 510. It is preferable to provide a door D3, configured to allow entrance and exit, in the control room 400.

Incidentally, the above-described layout is illustrative, and the present invention is not limited thereto. For example, the console 27 and the projector control apparatus 200 may be installed in another room differently form the projector 100 although the projector 100, the projector control apparatus 200, and the console 27 are installed in the control room 400 in the above-described layout. In addition, the projector 100 may be provided in the examination room 300 as long as the projector 100 can be formed using a material that is not affected by a magnetic field. In addition, a machine room or the like, which is configured to install the gradient power supply 21 and the receiving circuitry 25 therein, may be provided in addition to the examination room 300 and the control room 400.

Next, an exterior appearance of the gantry 11 will be described with reference to FIG. 4. FIG. 4 is a perspective view of the gantry housing 51 according to the present embodiment. As illustrated in FIG. 4, the hollow bore 53 is formed in the gantry housing 51. A rail 55, which is parallel to the central axis Z of the bore 53, is formed below the bore 53 of the gantry housing 51.

The rail 55 is a structure that guides slide of the couch top 131 and the movable screen apparatus 15 along the central axis Z. The rail 55 is provided on an inner wall 57 of the gantry housing 51 which is in contact with the bore 53. The rail 55 is formed using a non-magnetic material that does not act with a magnetic field used for the magnetic resonance imaging. Herein, a direction from the couch side toward the projector side in relation to the Z-axis is defined as a positive Z-axis direction and a direction from the projector side toward the couch side is defined as a negative Z-axis direction.

The exterior illuminator 56 is provided on an exterior surface of the gantry housing 51. The exterior illuminator 56 includes, for example, at least one light-emitting diode and an optical element. The exterior illuminator 56 may include a plurality of light-emitting diodes which generate light with a plurality of hues, respectively. At this time, the exterior illuminator 56 generates light having an arbitrary hue and arbitrary brightness based on the control of the system control circuitry 38 or operation of a switch or the like which is provided on the exterior surface of the gantry housing 51 or the like.

Incidentally, an arbitrary light-emitting device (for example, a fluorescent tube and the like that has a non-magnetic or magnetically shielded property) may be used as a light source of the exterior illuminator 56 instead of the light-emitting diode. The optical element reflects and diffuses the light generated by the light-emitting diode. The exterior illuminator 56 causes the exterior surface of the gantry housing 51 to be irradiated with the light generated by the light-emitting diode using the optical element.

The exterior illuminator 56 is installed on the exterior surface on the couch 13 side, for example, near an end (outer periphery) of the exterior surface so as to enclose a flare portion of the gantry housing 51. At this time, the exterior illuminator 56 has a substantially C-shape that exclude a connection portion between the gantry housing 51 and the couch 13 as illustrated in FIG. 4. The exterior illuminator 56 emits light toward the exterior surface of the gantry housing 51. The brightness and hue of the light generated by the exterior illuminator 56 is controlled by the system control circuitry 38. The control of the exterior illuminator 56 performed by the system control circuitry 38 will be described when describing the illumination state changing function 384.

The in-bore illuminator 58 is arranged on a surface of the rail 55, which is formed to be parallel to the central axis Z of the bore 53, in the lower part of the bore 53 of the gantry housing 51. For example, the in-bore illuminator 58 is arranged on a side portion 581 on an upper surface of the rail 55 along the central axis Z as illustrated in FIG. 4. Incidentally, the portion in which the in-bore illuminator 58 is provided is not limited to the lower part of the bore 53, the rail, and the like. In addition, the in-bore illuminator 58 may include an optical element that diffuses the light generated by the light-emitting diode. At this time, the in-bore illuminator 58 causes the inside of the bore 53 to be irradiated with the diffused light.

The in-bore illuminator 58 includes a plurality of light-emitting diodes along the central axis Z. The in-bore illuminator 58 may include the plurality of light-emitting diodes which generate light with a plurality of hues, respectively. At this time, the in-bore illuminator 58 generates light having an arbitrary hue and arbitrary brightness based on the control of the system control circuitry 38 or operation of a switch or the like which is provided on the exterior surface of the gantry housing 51 or the like. Incidentally, an arbitrary light-emitting device may be used as a light source of the in-bore illuminator instead of the light-emitting diode. The control of the in-bore illuminator 58 performed by the system control circuitry 38 will be described when describing the illumination state changing function 384.

The projector 59 is provided on the exterior surface of the gantry housing 51. To be specific, the projector 59 is provided in an opening portion of the bore 53 on the couch 13 side, that is, at an uppermost end of the bore 53 as illustrated in FIG. 4. Incidentally, the projector 59 may be provided in the flare portion of the gantry housing 51 on the couch 13 side or the like. The projector 59 emits light having a predetermined wavelength toward the rail 55 at the substantially lower side according to a user's instruction through the input circuitry 36. The light emitted from the projector 59 is used for positioning configured to move a part of a patient, which is a photographing target, to a center position of the magnetic field (referred to also as isocenter).

As illustrated in FIG. 4, the projector 59 emits the light (hereinafter, referred to as positioning light) having the predetermined wavelength with a predetermined width along the Z-axis and the Y-axis, for example. At this time, an intersection line 591 between a face defined by the positioning light along the Z-axis and a face defined by the positioning light along the Y-axis is positioned immediately below the projector 59. For example, the intersection line (hereinafter, referred to as intersection line light) 591 corresponds to a line segment which is parallel to the vertical direction and passes through the projector 59 in the bore 53.

The projector 59 causes the patient placed on the couch top 131 to be irradiated with the positioning light. At this time, a position of the patient illuminated by the intersection, that is, a position (hereinafter, referred to as a couch top position) of the couch top 131 at an input time of the positioning instruction are output to the system control circuitry 38 when a positioning instruction is input according to the user's instruction through the input circuitry 36. Next, the system control circuitry 38 controls the couch driving device 135 using the imaging control circuitry 31 in order to move the couch top position corresponding to a position designated by the user to the center position of the magnetic field in the bore 53. Accordingly, the part as the photographing target desired by the user is moved to the center position of the magnetic field in the bore 53.

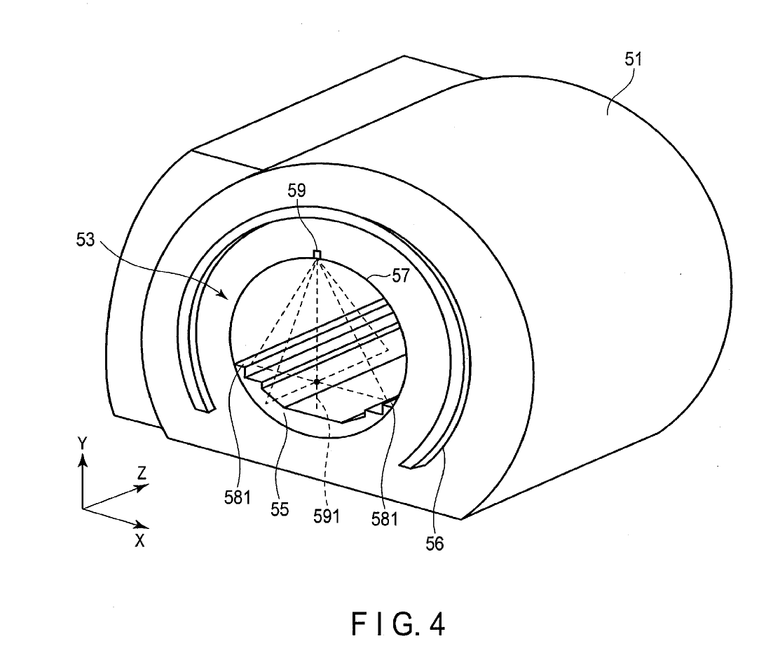

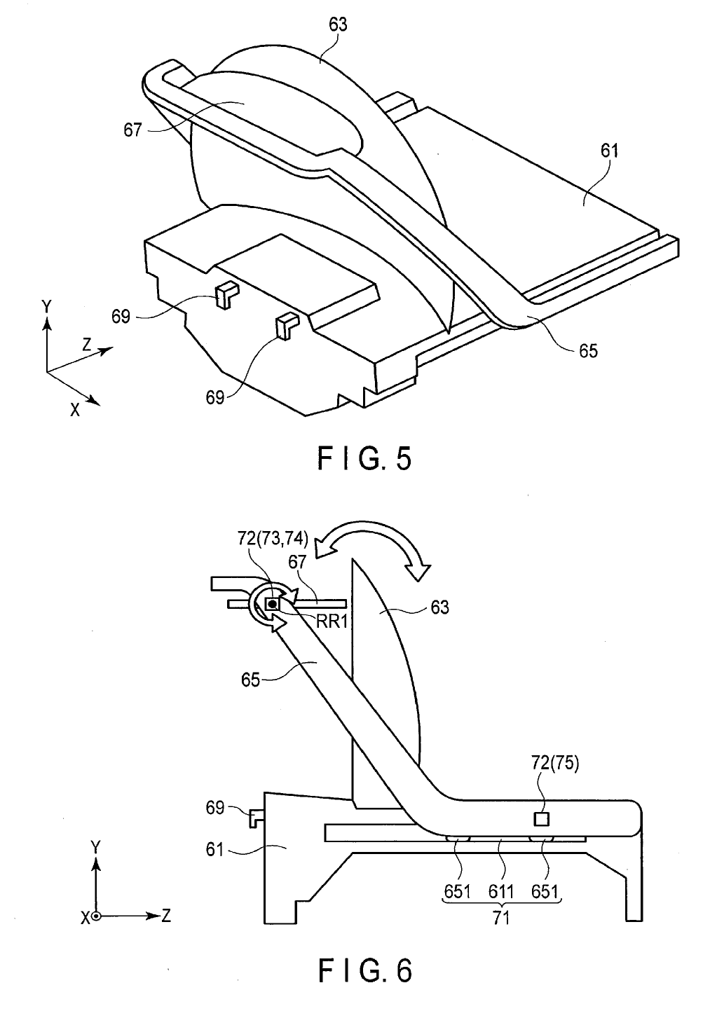

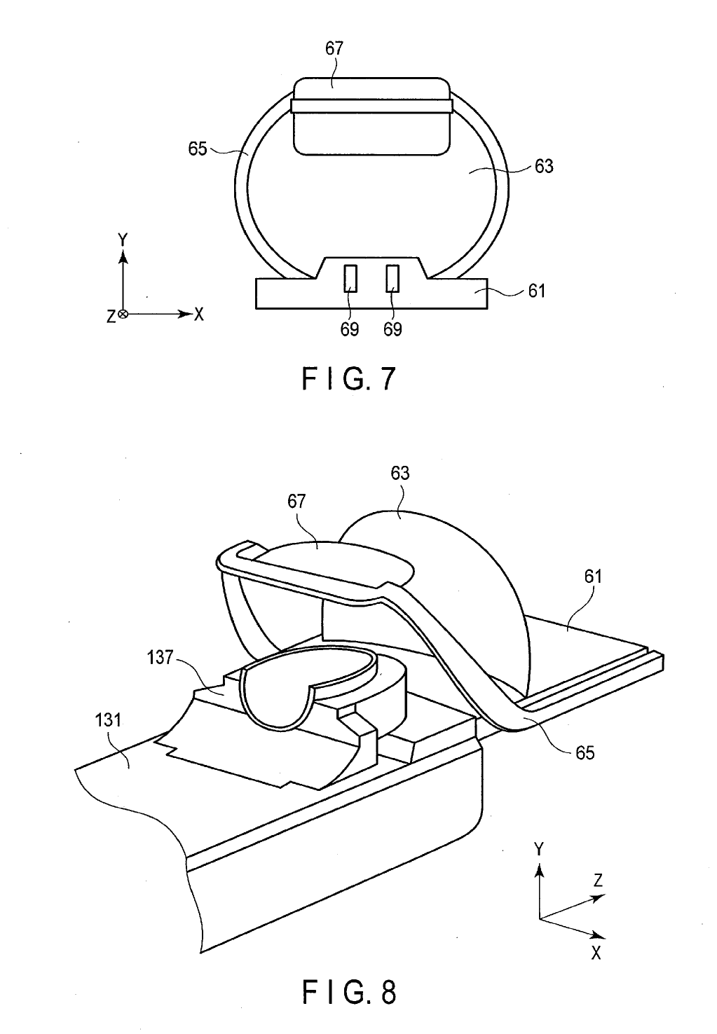

Next, a structure of the movable screen apparatus 15 will be described with reference to FIGS. 5 to 8. FIG. 5 is a perspective view of the movable screen apparatus 15 according to the present embodiment. FIG. 6 is a side view of the movable screen apparatus 15. FIG. 7 is a front view of the movable screen apparatus 15. FIG. 8 is a perspective view of the movable screen apparatus 15 and the couch top 131 which are couple with each other.

As illustrated in FIGS. 5 to 8, the movable screen apparatus 15 includes a movable carriage 61, a screen 63, a support arm 65, and a reflecting plate 67. The movable carriage 61 is a structural body that moves along the rail 55 provided on the inner wall 57 of the gantry housing 51. A wheel (not illustrated) to roll the rail 55 is attached to a lower part of the movable carriage 61 in order to enhance a traveling property of the rail 55.

Incidentally, the wheel is not necessarily provided as long as the movable carriage 61 can travel along the rail 55, and a face in contact with the rail 55 may be formed using a material having a low coefficient of friction. The movable carriage 61 and the rail 55 are formed to enable the movable carriage 61 to move from an end of the bore 53 on the couch 13 side (the negative Z-direction) to an end thereof on the projector 100 side (the positive Z-direction). A bottom face of the movable carriage 61 preferably has a shape that can be fit with the rail 55. When the movable carriage 61 and the rail 55 are engaged with each other, it is possible to make the rail 55 unnoticeable when the gantry 11 is viewed from the outside in a state in which the movable carriage 61 is arranged at the end of the bore 53. The movable carriage 61 supports the screen 63 and the support arm 65. The movable carriage 61 is formed using a non-magnetic material that does not act with a magnetic field such as resin.

As illustrated in FIG. 5, a coupling portion 69, configured for coupling with the couch top 131, is formed in the movable carriage 61. As illustrated in FIG. 8, the movable carriage 61 and the couch top 131 are coupled using the coupling portion 69. A patient fixing tool 137 is attached to a front portion (side in the positive Z-axis direction) of the couch top 131. The patient fixing tool 137 fixes a head of the patient P placed on the couch top 131. The patient fixing tool 137 has a curved shape so as to be capable of covering an occipital part without interrupting the field of view of the patient P placed on the back on the couch top 131. That is, a sincipital-part side of the patient fixing tool 137 is opened. Accordingly, the patient fixing tool 137 can mitigate the sense of blockage of the patient P, and further, mitigate constriction of the field of view of the patient P as compared to a fixing portion that covers the entire head. The patient fixing tool 137 is integrally molded using a non-magnetic material such as resin using a mold having the above-described shape, for example.

As illustrated in FIGS. 5 to 8, the screen 63 is installed upright on the movable carriage 61. The image from the projector 100 (not illustrated) is projected on the screen 63. The screen 63 is provided to be tiltable with respect to the movable carriage 61. To be specific, the movable carriage 61 is provided to be tiltable by a tilting mechanism (not illustrated) provided therein. The screen 63 is held to be perpendicular or have a predetermined gradient angle with respect to a front surface of the movable carriage 61 by adjusting a tilted angle of the screen 63 with respect to the front surface of the movable carriage 61. As described above, the projector 100 is arranged on the opposite side of the couch 13 with the screen 63 interposed therebetween.

Herein, a face of the screen 63 on the projector 100 side is referred to as a rear surface, and a face thereof on the couch 13 side is referred to as the front surface. The screen 63 is preferably formed using a semitransparent material in order to allow the image to be projected on the front surface thereof. Semitransparent plastic, ground glass, or the like is preferably used as such a semitransparent material. When the screen 63 is formed using the semitransparent material, the rear surface of the screen is irradiated with the projected light emitted from the projector 100, and the image corresponding to the projected light is projected on the front surface. Accordingly, the patient P or the like can view the image, which is projected on the front surface, from the couch 13 side.

The screen 63 may be a model having a planar shape or a model having a curved shape. The screen 63 is preferably arranged such that a concave face thereof is directed to the couch 13 side, that is, forms the front surface in the case of having the curved shape. When the concave face is directed to the couch 13 side, it is possible to cover the perimeter of the rear side of the head of the patient P placed on the couch top 131 with the screen 63. Accordingly, it is possible to make the field of view of the patient P to be filled with the image projected on the screen 63 such that the patient P is immersed in the image.

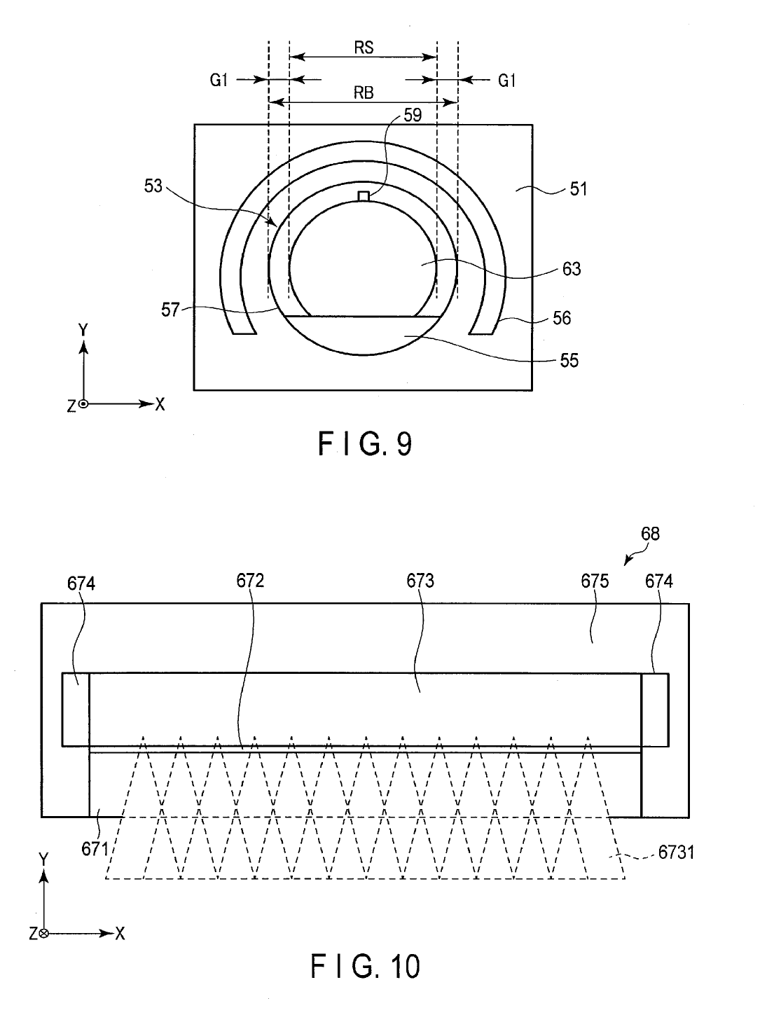

FIG. 9 is a schematic front view of the screen 63 which is arranged inside the bore 53. As illustrated in FIG. 9, the screen 63 has an outer diameter RS which is smaller than a diameter RB of the inner wall 57 in contact with the bore 53 of the gantry housing 51. It is possible to insert the movable screen apparatus 15 inside the bore 53, in that the outer diameter RS is designed to be smaller than the inner diameter RB in this manner.

Incidentally, a wind flows inside the bore 53 from a ventilation fan (not illustrated) provided in the gantry 11. When a gap G1 is provided between an edge of the screen 63 and the inner wall 57, it is possible to prevent the wind blown out from the ventilation fan from being interrupted by the screen 63. The outer diameter RS is preferably designed to be smaller than the inner diameter RB by, for example, 10 mm to 50 mm. In other words, the gap G1 is preferably designed to be 10 mm to 50 mm.

As illustrated in FIGS. 5 to 8, the support arm 65 is attached to the movable carriage 61. As will be described below, the support arm 65 is attached to the movable carriage 61 to be slidable in the Z-axis direction. The support arm 65 supports the reflecting plate 67 to be arranged in a space on the front surface side of the screen 63. Incidentally, the support arm 65 may support the reflecting plate 67 in a detachable manner. The reflecting plate 67 is, for example, a mirror.

The reflecting plate 67 is spaced apart from the front surface of the movable carriage 61 to a degree that prevents collision with the head of the patient P placed on the couch top 131 in a state in which the movable carriage 61 and the couch top 131 are coupled with each other, and is supported by the support arm 65. The support arm 65 has a shape that prevents the field of view of an external observer from being interrupted when the screen 63 is viewed from the outside of the gantry 11. The support arm 65 preferably has a semi-ring shape or a semi-saddle shape which includes an arcuate portion along a contour of the screen 63 in order not to interrupt the field of view of the external observer as illustrated in FIGS. 5 to 8. In this case, both ends of the support arm 65 are attached to the side portion of the movable carriage 61, and the support arm 65 is attached to the movable carriage 61 such that the arcuate portion of the support arm 65 is positioned in the space on the front surface side of the screen 63.

Incidentally, the shape of the support arm 65 is not limited to the above-described semi-ring or semi-saddle shape, but may have any shape as long as the reflecting plate 67 can be arranged in the space on the front surface side of the screen 63. For example, the support arm 65 may be configured using a pair of arms each of which has substantially a rod shape. In this case, it is preferable when one end of the pair of arms is attached to both side portions of the movable carriage 61 and the other end thereof is attached to the reflecting plate 67.

As illustrated in FIGS. 5 to 8, the reflecting plate 67 is provided at a substantially uppermost part of the support arm 65. Incidentally, a reflection unit may also be provided instead of the reflecting plate 67. The reflection unit includes a beam splitter having predetermined transmittance and reflectance, and includes a reflecting plate that reflects the image from the image source and a light source that irradiates the beam splitter with light. FIG. 10 is a cross-sectional view illustrating an example of a configuration of a reflection unit 68. The cross section of the reflection unit 68 in FIG. 10 corresponds to a face perpendicular to the Z-axis. Incidentally, FIG. 10 may illustrate a face perpendicular to the X-axis. As illustrated in FIG. 10, the reflection unit 68 is configured of a plurality of non-magnetic materials. To be specific, the reflection unit 68 includes a beam splitter 671, a film 672, a light guide plate 673, a light source 674, and a base cover 675.

The beam splitter 671 has a predetermined transmittance and a predetermined reflectance. The beam splitter 671 is a half mirror to which a dielectric film adheres, for example. When the back of the beam splitter 671 is brighter than the front of the beam splitter 671, the patient P can visually recognize the back of the beam splitter 671. On the contrary, when the back of the beam splitter 671 is darker than the front of the beam splitter 671, the beam splitter 671 functions as the mirror.

The film 672 is installed at the back of the beam splitter 671. The film 672 transmits light 6731 generated by the light guide plate 673. A predetermined image is provided in the film 672. Incidentally, the predetermined image may be printed on the film 672 or may be pasted thereto. Examples of the predetermined image include a photograph, advertisement, a logo mark, descriptions on directions relating to an examination, and the like. In addition, the film may be a monochromatic film instead of including the image.

The light guide plate (light guide) 673 is installed at the back of the film 672. That is, the light guide plate 673 is provided at the back of the beam splitter which is on a non-couch top side. The light guide plate 673 is configured using transparent resin, for example, an acrylic plate, polycarbonate or the like. A front surface of the light guide plate 673 is subjected to processing that corresponds to a pattern design configured to realize a desired light emitting pattern. The light guide plate 673 emits light using the light generated by the light source 674. The light 6731 generated by the light guide plate 673 passes through the film 672 and the beam splitter 671 and is emitted to the outside of the reflection unit 68.

The light source 674 is arranged at at least one end of the light guide plate 673. Preferably, the light source 674 is arranged around the light guide plate 673. The light source 674 is, for example, a light-emitting diode. Incidentally, the light source 674 may be an arbitrary light-emitting device without being limited to the light-emitting diode. Turning ON and OFF of the light source 674 is controlled by the system control circuitry 38. The control of ON and OFF of the light source 674 will be described later when describing a reflection state changing function 382.

As illustrated in FIG. 10, the base cover 675 is a base material that encloses and supports the beam splitter 671, the film 672, the light guide plate 673, and the light source 674. A rotation shaft RR1, which is rotatably connected to the support arm 65, is provided in two opposing side faces of the base cover 675.

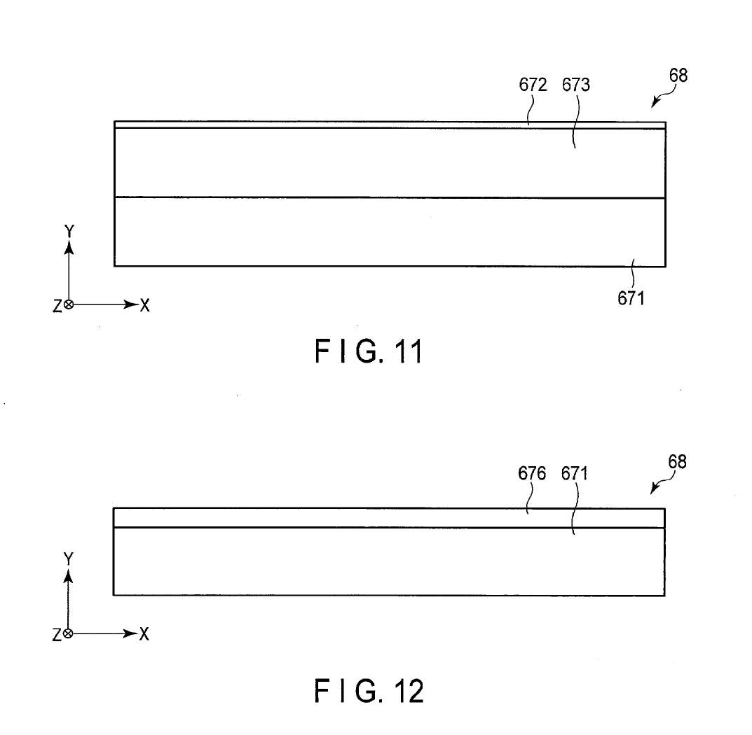

FIG. 11 is a cross-sectional view illustrating an example of the reflection unit 68 having a different configuration from that of FIG. 10. FIG. 11 does not illustrate the light source 674 and the base cover 675. A difference between FIG. 11 and FIG. 10 is that the light guide plate 673 is provided at the back of the beam splitter 671, and the film 672 is provided on a back side of the light guide plate 673. When the reflection unit 68 has the configuration as illustrated in FIG. 11, the film 672 can be configured using a material having optical non-transmissibility. That is, the optical transmissibility does not matter in terms of the material of the film 672 in the configuration of the reflection unit 68 in FIG. 11.

FIG. 12 is a cross-sectional view illustrating an example of the reflection unit 68 having a different configuration from those of FIGS. 10 and 11. FIG. 12 does not illustrate the base cover 675. A difference of FIG. 12 from FIGS. 10 and 11 is that a monitor (hereinafter, referred to as a back monitor) 676 is installed at the back of the beam splitter 671. At this time, a predetermined image or a predetermined moving image is displayed on the back monitor 676. The back monitor 676 is controlled by the system control circuitry 38. The control of the back monitor 676 will be described when describing the reflection state changing function 382. Hereinafter, various descriptions relating to the reflecting plate 67 can be applied to the reflection unit 68.

The reflecting plate 67 is rotatably provided in the support arm 65 in order to manually adjust an angle of the reflecting plate 67 in accordance with an angle according to the patient P. Incidentally, the reflecting plate 67 may be supported by the support arm 65 in a detachable manner. To be specific, the reflecting plate 67 is provided to be rotatable about the rotation shaft RR1 by a rotating mechanism (not illustrated) which is provided in the support arm 65.

The rotation shaft RR1 is provided to be parallel to the X-axis so as to be capable of adjusting a direction of the reflecting plate 67 with respect to the front surface of the screen 63, for example. To be more specific, the support arm 65 is preferably provided, at least, to be switchable between a first angle for a first projection format and a second angle for a second projection format which will be described later.

The first projection format is a format to view the image of the screen 63 without the intervention of the reflecting plate 67 from the outside of the gantry 11. Thus, the first angle of the reflecting plate 67 according to the first projection format is preferably set to be, for example, substantially horizontal to an angle that does not interrupt the field of view of the patient P or the like present at the outside of the gantry 11. The second projection format is a format to view the image through the reflecting plate 67 inside the bore 53. Thus, the second angle of the reflecting plate 67 according to the second projection format is preferably set to an arbitrary angle between horizontal and vertical angles according to physique or the like of the patient P serving as an observer. The patient P whose head is arranged in the patient fixing tool 137 can view the image projected on the screen 63 through the reflecting plate 67 arranged at the second angle.

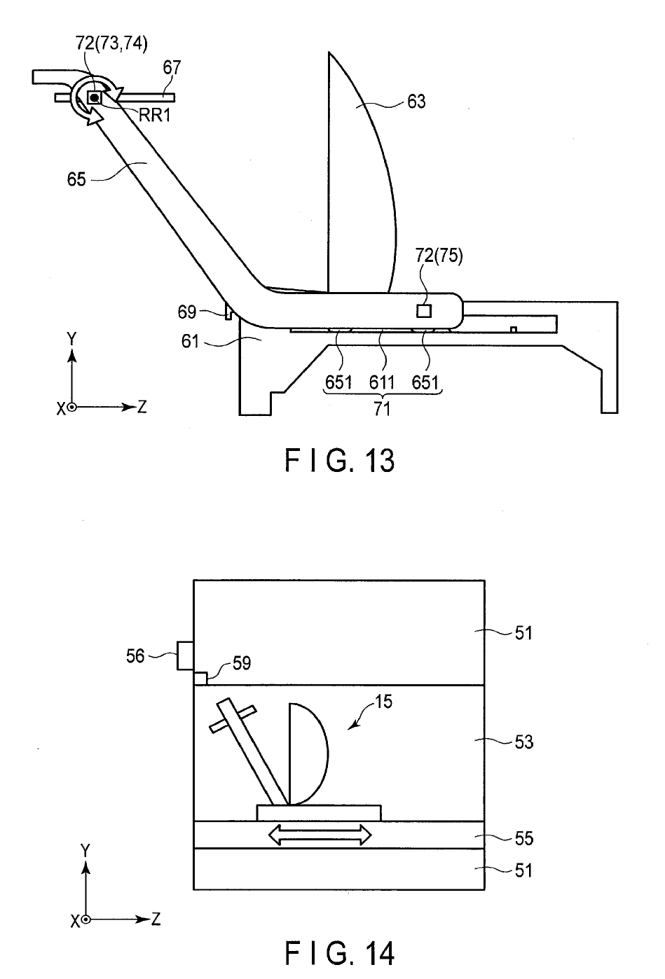

A sliding mechanism 71 of the support arm 65 is preferably provided in the movable carriage 61 in order to adjust the position of the reflecting plate 67 in relation to the Z-axis. FIG. 13 is a diagram illustrating a side face of the movable screen apparatus 15 in which the support arm 65 illustrated in FIG. 6 is slid in relation to the Z-axis. As illustrated in FIGS. 6 and 13, the sliding mechanism 71 includes a guide 611 which guides the slide of the support arm 65 along the Z-axis is formed in the movable carriage 61. The guide 611 is provided along the Z-axis on both the side faces of the movable carriage 61 in order to avoid contact with the support arm 65 and the screen 63.

The guide 611 may be realized in any mode, and, for example, is realized by a void provided on the side face of the movable carriage 61 along the Z-axis. As illustrated in FIGS. 6 and 13, a base portion of the support arm 65 that faces the guide 611 is preferably provided with a wheel 651 in order to enhance the mobility of the support arm 65.

When the sliding mechanism 71 is provided, a medical professional such as a doctor, a technician, and a nurse, the patient P and the like can cause the reflecting plate 67 to approach or be spaced apart from the screen 63 by pushing or pulling the support arm 65 in the Z-axis direction. Accordingly, it is possible to adjust the position of the reflecting plate 67 in relation to the Z-axis direction.

Incidentally, the sliding mechanism 71 is realized using the guide 611 provided in the movable carriage 61 and the wheel 651 provided in the support arm 65 in the above description. However, the present embodiment is not limited thereto. Any mechanism may be used as the sliding mechanism 71 according to the present embodiment as long as the support arm 65 is relatively slidable with respect to the movable carriage 61. For example, it may be configured such that a guide is provided in the support arm 65 along the Z-axis, and a wheel to travel the guide is provided in the movable carriage 61. In addition, the sliding mechanism 71 may be realized using a ball screw, a slide rail, or the like.

FIG. 14 is a simple side view of the movable screen apparatus 15 which is arranged in the bore 53 of the gantry 11. As illustrated in FIG. 14, the movable carriage 61 of the movable screen apparatus 15 is slidably provided in the rail 55. Typically, a driving device is not mounted to the movable screen apparatus 15. The movable screen apparatus 15 slides to be interlocked with slide of the couch top 131 caused by the couch driving device 135. Incidentally, the movable screen apparatus 15 can also slide in relation to the Z-axis by being pushed and pulled by the patient P, a medical professional or the like.

Through the above-described configuration, the medical image diagnostic apparatus 10 according to the present embodiment can realize the first projection format, which projects the image on the movable screen apparatus 15 in a state in which the movable screen apparatus 15 is arranged at the end of the bore 53 on the couch side, and the second projection format which projects the image on the movable screen apparatus 15 in a state in which the couch top 131 and the movable screen apparatus 15 are coupled with each other.

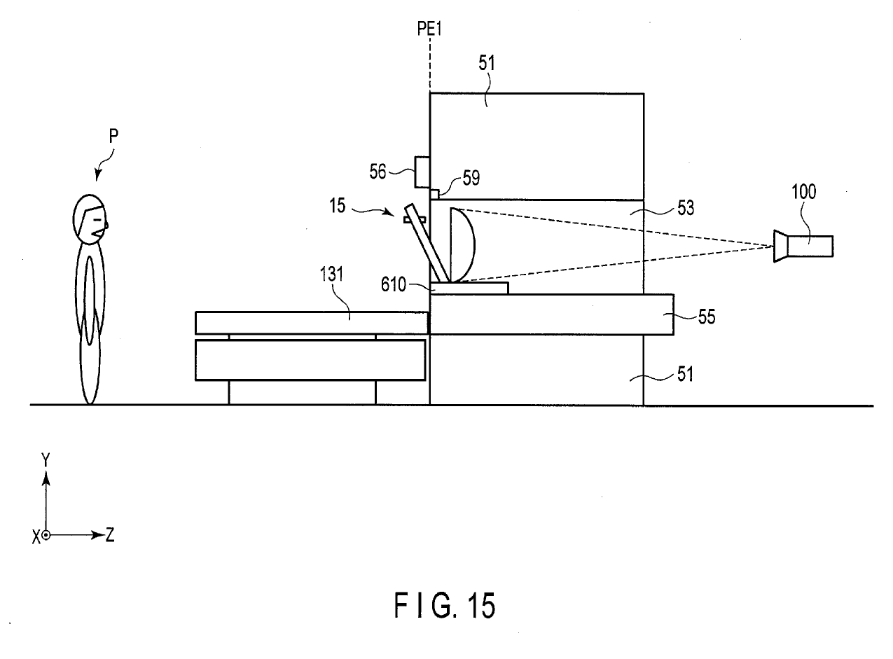

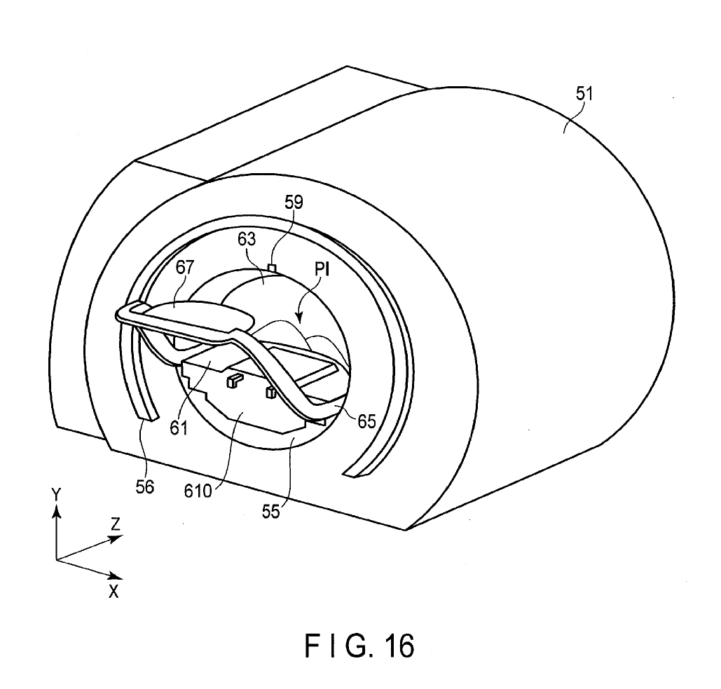

FIG. 15 is a diagram illustrating the movable screen apparatus 15 in the first projection format from a lateral side of the gantry 11. FIG. 16 is a diagram illustrating the movable screen apparatus 15 in the first projection format from a front side of the gantry 11. As illustrated in FIGS. 15 and 16, the movable screen apparatus 15 is arranged such that the screen 63 is positioned at the end of the bore 53 on the couch side in the first projection format. The patient P or a medical professional view an image PI, which is projected on the screen 63 from the outside of the gantry housing 51 without the intervention of the reflecting plate 67 in the first projection format. Since the screen 63 is arranged at the end on the couch side, the screen 63 occludes the bore 53 and it is possible to prevent the patient P from viewing the inside of the bore 53.

In addition, since the image PI is projected on the screen 63, the bore 53 blunts the visual recognition of the patient P on being present inside the examination space, and it is possible to relieve a sense of fear against entering the bore 53. Hereinafter, a positioning mode of the gantry 11, the couch 13, and the movable screen apparatus 15 for the first projection format is referred to as a first positioning mode.

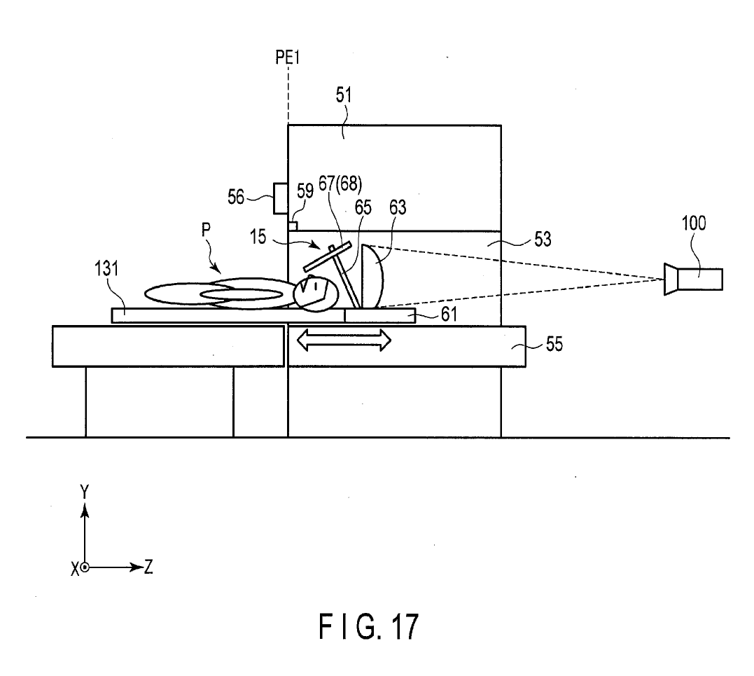

FIG. 17 is a diagram illustrating the movable screen apparatus 15 in the second projection format from a lateral side of the gantry 11. As illustrated in FIG. 17, the patient P views the image projected on the front surface of the screen 63 through the reflecting plate 67 in the state of being placed on the couch top 131 and inserted into the bore 53 in the second projection format. Since the couch top 131 and the movable screen apparatus 15 are coupled with each other, a distance between the patient P and the screen 63 is kept constant regardless of the slide of the movable screen apparatus 15 in the Z-axis direction. Accordingly, it is possible to enhance the sense of immersion into the image projected on the screen 63 and to relieve the sense of blockage inside the bore 53. Hereinafter, a positioning mode of the gantry 11, the couch 13, and the movable screen apparatus 15 for the second projection format is referred to as a second positioning mode.

The movable screen apparatus 15 includes a detector 72 that detects a positioning state of the reflecting plate 67 with respect to the screen 63 or a changing operation of this positioning state. The detector 72 detects a change of a relative position of the support arm 65 or the reflecting plate 67 with respect to the screen 63 at the time of transition from the first positioning mode to the second positioning mode and at the time of transition from the second positioning mode to the first positioning mode.

The positioning state of the reflecting plate 67 with respect to the screen 63 indicates a state in which the reflecting plate 67 is arranged at the first angle or the second angle in the first positioning mode. Incidentally, the positioning state of the reflecting plate 67 may be a state in which the reflecting plate 67 is attached to the support arm 65 or a state in which the reflecting plate 67 is detached from the support arm 65. In addition, there is also a case in which the positioning state of the reflecting plate 67 is changed along with the movement of the support arm 65 with respect to the screen 63.

The changing operation of the positioning state of the reflecting plate 67 corresponds to an operation to change an angle of the reflecting plate 67 from the first angle to the second angle in the first positioning mode or an operation to change the angle thereof from the second angle to the first angle. Incidentally, the changing operation of the positioning state of the reflecting plate 67 may be attachment and detachment operations of the reflecting plate 67 with respect to the support arm 65. In addition, the changing operation of the positioning state of the reflecting plate 67 may be a movement operation of the support arm 65 with respect to the screen 63.

To be specific, the detector 72 detects the positioning state of the reflecting plate 67 or the changing operation at a timing of an operation start time of the reflecting plate 67 or the support arm 65 or an attachment time of the reflecting plate 67 to the support arm 65. Incidentally, the detector 72 may perform the detection at a timing during an operation period of the reflecting plate 67 or the support arm 65. In addition, the detector 72 detects the positioning state of the reflecting plate 67 or the changing operation at a timing of an operation end time of the reflecting plate 67 or the support arm 65 or a detachment time of the reflecting plate 67 from the support arm 65. Incidentally, the detector 72 may detect the positioning state of the reflecting plate 67 or the changing operation at any two or more timings of the operation start time, the attachment time, the time during the operation period, and the operation end time.

The detector 72 detects at least one of the attachment and detachment of the reflecting plate 67 to and from the support arm 65, the movement of the support arm 65 with respect to the screen 63, and the rotation of the reflecting plate 67 with respect to the rotation shaft RR1. The detector 72 is configured using a non-magnetic material. The detector 72 is realized by any one of an optical sensor, a mechanical sensor, an electric sensor having a non-magnetic property, and a magnetic sensor having a magnetically shielded property, or a combination of these sensors. Incidentally, the detector 72 may be provided at a position independent from the rotation shaft RR1.

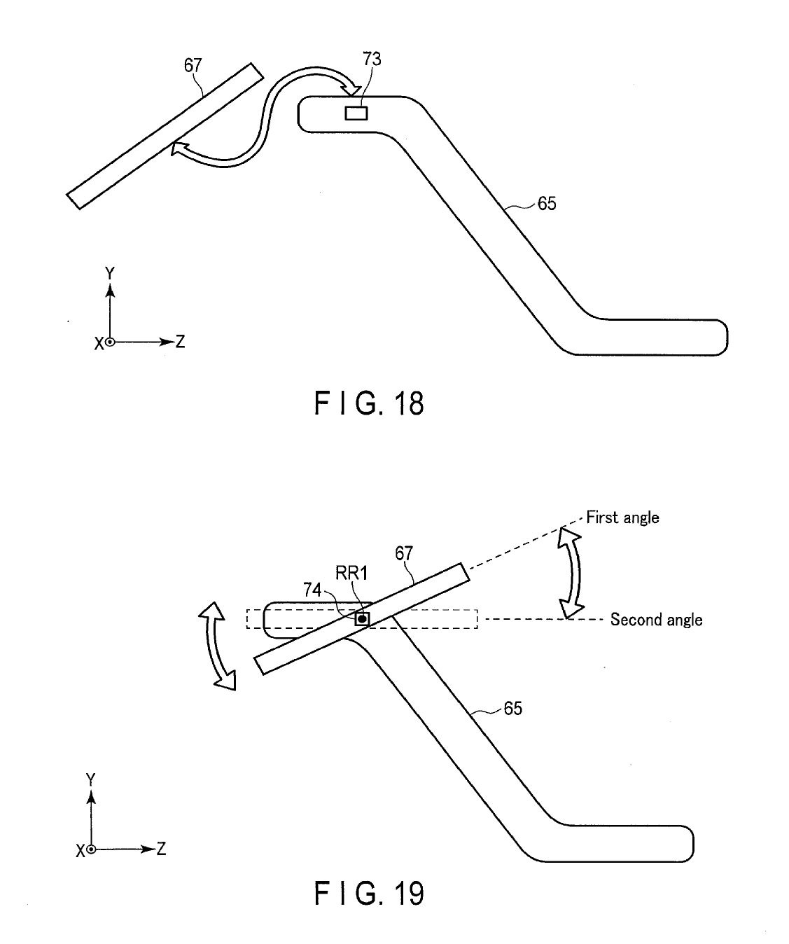

FIG. 18 is a diagram illustrating an example of the attachment and detachment of the reflecting plate 67 to and from the support arm 65. The detector (hereinafter, referred to as an attachment and detachment detector 73), which detects the attachment and detachment of the reflecting plate 67 to and from the support arm 65, is provided at a position adjacent to the rotation shaft RR1 in the support arm 65. The attachment and detachment detector 73 is a contact sensor, for example. The attachment and detachment detector 73 detects the time (the attachment time and the detachment time) of the attachment and detachment of the reflecting plate 67 to and from the support arm 65. The attachment and detachment detector 73 outputs the attachment time and the detachment time to the system control circuitry 38. As illustrated in FIG. 18, the attachment and detachment detector 73 detects the attachment and detachment of the reflecting plate 67 to and from the support arm 65.

FIG. 19 is a diagram illustrating an example of a rotating operation of the reflecting plate 67 about the rotation shaft RR1. The detector (hereinafter, referred to as a rotation detector 74), which detects the rotation of the reflecting plate 67 with respect to the rotation shaft RR1, is provided at a position adjacent to the rotation shaft RR1 in the support arm 65. Examples of the rotation detector 74 include a rotation angle sensor, a position sensor, an acceleration sensor, and the like. As illustrated in FIG. 19, the rotation detector 74 detects the rotating operation of the reflecting plate 67.

Examples of the rotation angle sensor include a rotary encoder, a potentiometer, and the like. The rotation angle sensor detects a rotation angle (the first angle and the second angle) of the reflecting plate 67 about the rotation shaft RR1. Examples of the position sensor include a position sensitive detector, a limit switch, and the like. The position sensor detects a position of the reflecting plate 67 about the rotation shaft RR1. Incidentally, the rotation angle sensor and the position sensor may be a mechanical sensor that is provided in a rotating mechanism such as a freely rotatable ratchet provided in the rotation shaft RR1.

That is, the rotation angle sensor and the position sensor detects the relative position of the reflecting plate 67 with respect to the screen 63. Accordingly, the rotation angle sensor and the position sensor detect a start time of the rotating operation of the reflecting plate 67 about the rotation shaft RR1, a period of rotation of the reflecting plate 67 about the rotation shaft RR1, and an end time of the rotating operation of the reflecting plate 67 about the rotation shaft RR1. The rotation angle sensor and the position sensor output the operation start time, the operation period, the operation end time relating to the rotating operation of the reflecting plate 67 to the system control circuitry 38.

The acceleration sensor is realized by, for example, a micro electro mechanical system (MEMS). The acceleration sensor detects the acceleration relating to the rotating operation of the reflecting plate 67 about the rotation shaft RR1. That is, the acceleration sensor detects the start time of the rotating operation of the reflecting plate 67 and the operation end time of the rotating operation of the reflecting plate 67 about the rotation shaft RR1. The acceleration sensor outputs the operation start time and the operation end time relating to the rotating operation of the reflecting plate 67 to the system control circuitry 38.

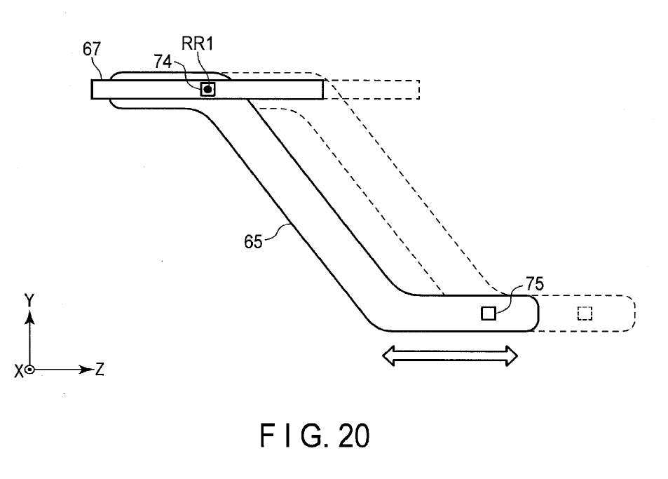

FIG. 20 is a diagram illustrating an example of the movement (slide) of the support arm 65 with respect to the screen 63. The detector (hereinafter, referred to as a movement detector 75), which detects the movement of the support arm 65 with respect to the screen 63, is provided in the support arm 65 or the movable carriage 61 to be adjacent to the sliding mechanism 71. At this time, the detector 72 detects the movement of the support arm 65 with respect to the screen 63 as detection of the positioning state of the reflecting plate 67 with respect to the screen 63 or detection of the changing operation of this positioning state. Examples of the movement detector 75 include a displacement sensor, a position sensor, an acceleration sensor, and the like. The displacement sensor is realized by a differential transformer, a linear encoder, and the like. As illustrated in FIG. 20, the movement detector 75 detects the movement of the support arm 65 caused by the sliding mechanism 71.

The displacement sensor detects displacement (movement) of the relative position of the support arm 65 with respect to the movable carriage 61, that is, displacement of the relative position of the reflecting plate 67 with respect to the screen 63. In addition, the position sensor detects the relative position of the support arm 65 with respect to the movable carriage 61. Accordingly, the displacement sensor and the position sensor detect a start time of the movement of the support arm 65 with respect to the screen 63, an end time of the movement of the support arm 65, and an operation period of the movement of the support arm 65. The displacement sensor and the position sensor output the start time, the end time, and the operation time of the movement of the support arm 65 to the system control circuitry 38.

The acceleration sensor detects the acceleration relating to the movement of the support arm 65. Incidentally, the acceleration sensor may detect acceleration of the wheel 651 accompanying movement of the wheel 651 in the guide 611. Accordingly, the acceleration sensor detect an operation start time of the support arm 65 accompanying the movement of the support arm 65 and an operation end time of the support arm 65. The acceleration sensor outputs the operation start time and the operation end time of the support arm 65 to the system control circuitry 38.

FIGS. 6 and 13 are diagrams illustrating examples of the attachment and detachment detector 73, the rotation detector 74, and the movement detector 75 which are provided in the support arm 65. As illustrated in FIGS. 6 and 13, the attachment and detachment detector 73 and the rotation detector 74 are provided in the support arm to be adjacent to the rotation shaft RR1. In addition, the movement detector 75 is provided in the support arm 65 to be near the sliding mechanism 71. The attachment and detachment detector 73, the rotation detector 74, and the movement detector 75 are connected to the system control circuitry 38 and the like via a predetermined cable (not illustrated).

(State Changing Function)

The state changing function 381 is a function of controlling each unit based on a positioning state of the reflecting plate 67 with respect to the screen 63 and a changing operation of this positioning state in order to change at least one state of an illumination state and an acoustic state of the space in which the movable screen apparatus 15 is arranged, the examination room 300 and the like, a photographing state to optically photograph the inside of the bore 53, and a reflection state of the reflecting plate 67. Hereinafter, various state changing functions according to the state changing function 381 will be described.

(Reflection State Changing Function)

The reflection state changing function 382 is a function of changing the reflection state of the reflection unit 68 based on a detection result from the detector 72. Hereinafter, the detector 72 is assumed as the rotation detector 74. A description will be given later regarding a case in which the detector 72 includes the attachment and detachment detector 73 and the rotation detector 74 (first Application Example) and a case in which the detector 72 includes the rotation detector 74 and the movement detector 75 (second application example). Incidentally, the reflection state changing function 382 can be executed along with the reflection state changing function 382 according to another application example and at least one function of other various state changing functions.

The system control circuitry 38 that realizes the reflection state changing function 382 controls supply of power to the light source 674 according to the angle of the reflection unit 68 with respect to the screen 63 in order to switch an ON state and an OFF state of the light source 674. To be specific, the system control circuitry 38 controls the supply of power to the light source 674 such that the light source 674 is turned ON, that is, the light source 674 is turned into the ON state when the first angle is detected as the positioning state of the reflection unit 68 by the rotation detector 74. For example, the system control circuitry 38 supplies current to the light source 674 in response to the detection of the first angle. At this time, the system control circuitry 38 maintains the supply of current to the light source 674 until detecting the second angle.

Incidentally, the system control circuitry 38 may control the supply of power to the light source 674 such that the light source 674 is turned ON over an operation period of a rotating operation from the second angle to the first angle and a period in which the first angle is detected in response to a start time of the rotating operation from the second angle to the first angle. In addition, the system control circuitry 38 may control the supply of power to the light source 674 such that the light source 674 is turned ON in response to an end time of the rotating operation from the second angle to the first angle.

The system control circuitry 38 controls the supply of power to the light source 674 such that the light source 674 is turned OFF, that is, the light source 674 is turned into the OFF state when the system control circuitry 38 detects the second angle as the positioning state of the reflection unit 68 using the rotation detector 74. For example, the system control circuitry 38 cuts off the supply of current to the light source 674 in response to the detection of the second angle.

Incidentally, the system control circuitry 38 may control the supply of power to the light source 674 such that the light source 674 is turned OFF over the operation period of the rotating operation from the first angle to the second angle and a period in which the second angle is detected in response to a start time of the rotating operation from the first angle to the second angle. In addition, the system control circuitry 38 may control the supply of power to the light source 674 such that the light source 674 is turned OFF in response to an end time of the rotating operation from the first angle to the second angle.

When the reflection unit 68 is configured of the beam splitter 671 and the back monitor 676, the back monitor 676 becomes a control target of the system control circuitry 38 in the reflection state changing function 382. At this time, the system control circuitry 38 controls the back monitor 676 according to the angle of the reflection unit 68 with respect to the screen 63 in order to switch an ON state and an OFF state of the back monitor 676. The ON state of the back monitor 676 corresponds to a display state of an image (moving image or the like) using the back monitor 676, and the OFF state of the back monitor 676 corresponds to a non-display state of the image using the back monitor 676.

The control of the back monitor 676 performed by the system control circuitry 38 is the same as the above-described control with respect to the light source 674, and thus, the description thereof will be omitted. The control of the back monitor 676 regarding each of the first application example and the second application example is the same as the control with respect to the light source 674, and thus, the description thereof will be omitted.

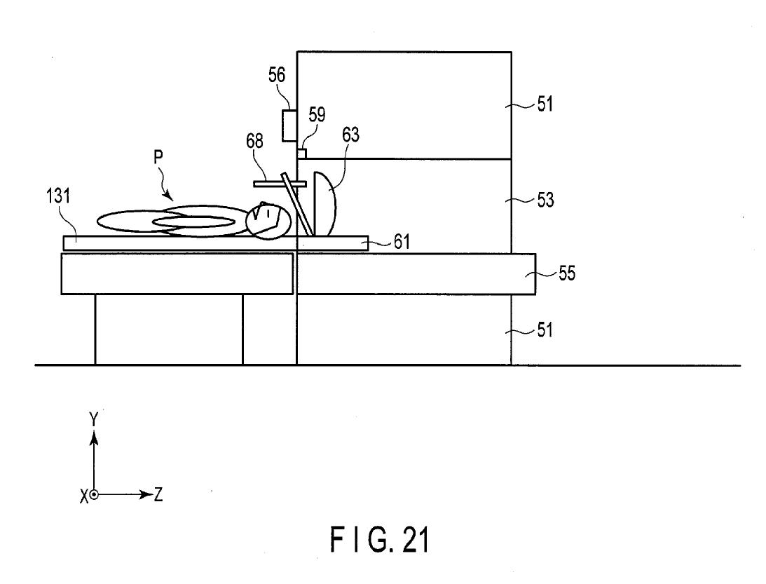

FIG. 21 is a diagram illustrating a patient placed on the couch top 131 before being inserted into the bore 53 and the movable screen apparatus 15 arranged at the end of the bore 53 on the couch 13 side from the lateral side of the gantry 11. As illustrated in FIG. 21, the reflection unit 68 is arranged at the first angle (to be substantially horizontal) at the front of the head of the patient P. At this time, the detector 72 detects the first angle.

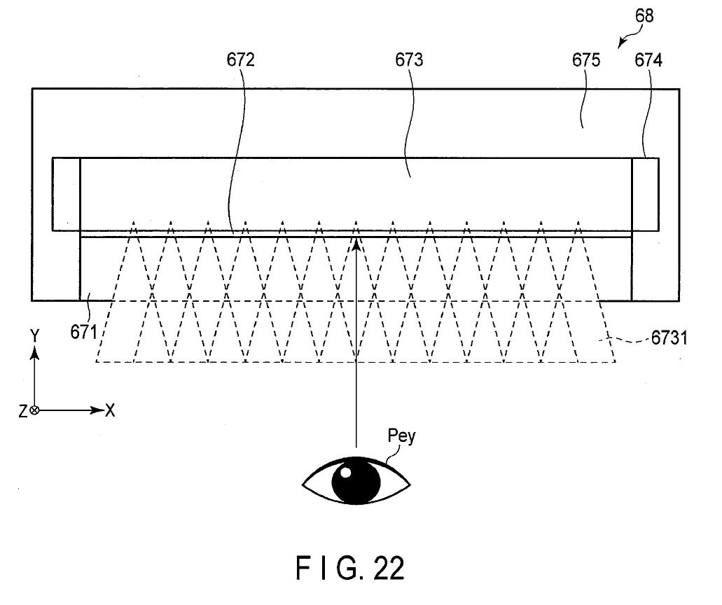

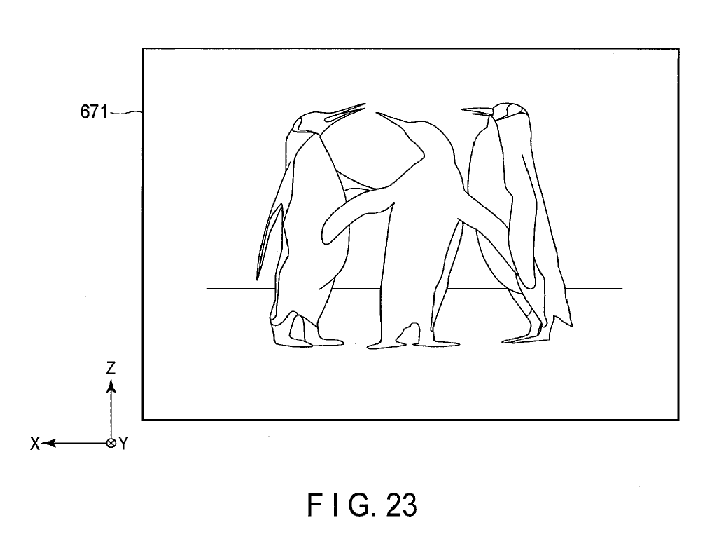

FIG. 22 is a diagram schematically illustrating the reflection unit 68 arranged at the first angle in FIG. 21 and an eye Pey of the patient P. FIG. 23 is a diagram illustrating an example of an image of the film 672 which is arranged at the back of the beam splitter 671 in FIGS. 21 and 22. The light source 674 is turned ON in each positioning state of the reflection unit 68 in FIGS. 21 and 22. Accordingly, the back of the beam splitter 671 becomes brighter than the front of the beam splitter 671. Thus, the patient P can visually recognize the image or the image of the film 672 arranged at the back of the beam splitter 671, directly as illustrated in FIG. 23.

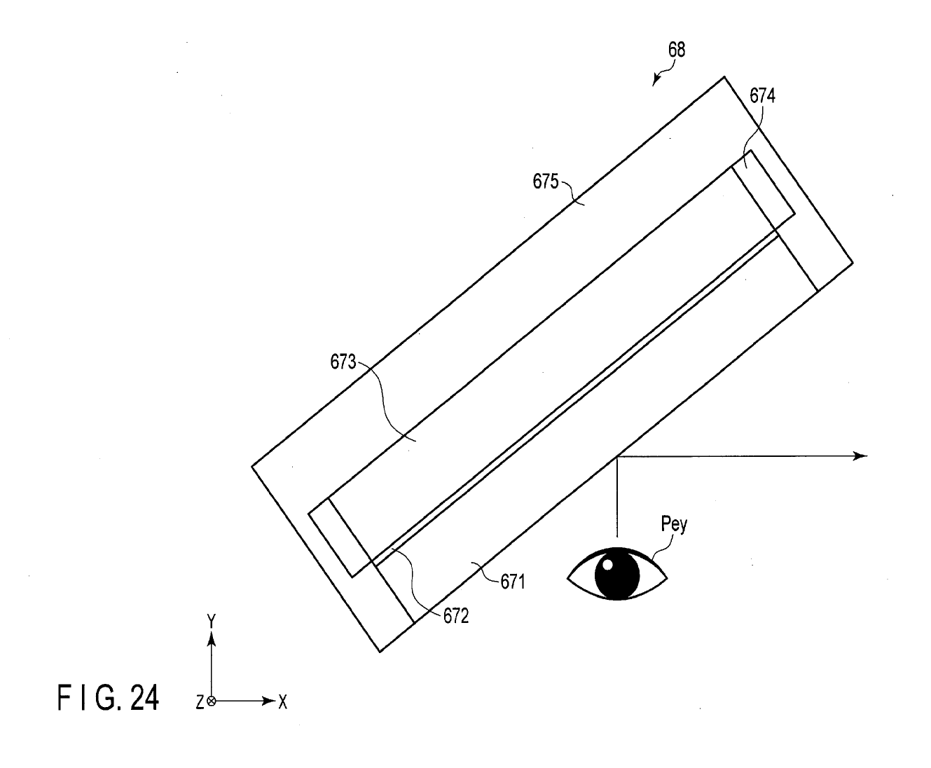

FIG. 24 is a diagram schematically illustrating the reflection unit 68 arranged at the second angle and the eye Pey of the patient P. The light source 674 is turned OFF in a positioning state of the reflection unit 68 in FIG. 24. Accordingly, the back of the beam splitter 671 becomes darker than the front of the beam splitter 671. Thus, the patient P can visually recognize the image of the screen 63 at a reflection destination of the beam splitter 671 through the reflection of the beam splitter 671 as illustrated in FIG. 24.

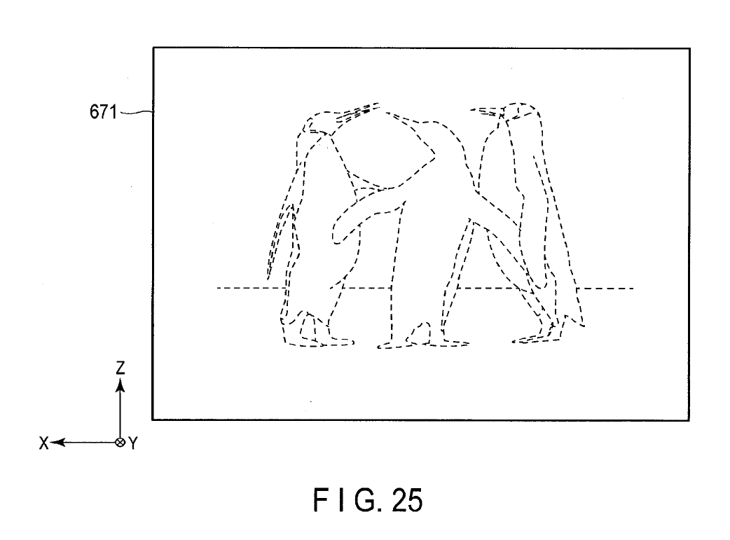

FIG. 25 is a diagram illustrating an example of an image of the film 672 which is arranged at the back of the beam splitter 671 in FIG. 24. As illustrated in FIG. 25, the image of the film 672 arranged at the back of the beam splitter 671 is positioned at the back of the image of the screen 63. In addition, the back of the beam splitter 671 becomes darker than the front of the beam splitter 671, and thus, the patient P is practically incapable of visually recognizing the image of the film 672.

According to the above-described configuration, it is possible to obtain effects to be described as follows.

According to the magnetic resonance diagnostic apparatus 10 of the present embodiment, which realizes the reflection state changing function 382, the beam splitter 671 having the predetermined transmittance and reflectance is provided, and it is possible to control the light source 674 according to the positioning of the reflecting plate 67, which reflects the image from the image source of the projector 100, and the reflection unit 68, which includes the light source 674 that irradiates the beam splitter 671 with light, with respect to the image source. That is, it is possible to switch the ON state and the OFF state of the light source 674 or the back monitor 676 mounted to the reflection unit 68 according to the angle or position of the reflection unit 68 with respect to the screen 63. Accordingly, it is possible to set the back of the beam splitter 671 to be brighter than the front of the beam splitter 671 in the reflection unit 68 arranged at the first angle. In addition, it is possible to set the back of the beam splitter 671 to be darker than the front of the beam splitter 671 in the reflection unit 68 arranged at the second angle.

In this manner, a face of the patient P is practically not reflected on the reflection unit 68 arranged at the first angle, that is, in the substantially horizontal state according to the magnetic resonance diagnostic apparatus according to the embodiment 10, the patient P can visually recognize the image of the film 672 arranged at the back of the beam splitter 671 or the image (moving image) of the back monitor 676 directly instead of visually recognizing the face through the reflection unit 68. Accordingly, it is possible to reduce the sense of discomfort of the patient P.

In the above-described manner, according to the magnetic resonance diagnostic apparatus 10, it is possible to provide environment in which the reflection unit 68 arranged at the first angle is effectively utilized by allowing the patient P to visually recognize the image of the film 672 arranged at the back of the beam splitter 671 or the image of the back monitor 676 in the reflection unit 68 arranged at the first angle, the anxiety of the patient P is improved without causing the uncomfortable feeling to the patient P, and the patient P can relax.

(First Application Example)

The first application example corresponds to a case in which the detector 72 further includes the attachment and detachment detector 73. The system control circuitry 38 that realizes the reflection state changing function 382 controls the supply of power to the light source 674 according to the attachment and detachment of the reflection unit 68 to and from the support arm 65 and the angle of the reflection unit 68 in order to switch the ON state and the OFF state of the light source 674. In the following description, redundant parts of the above description on the reflection state changing function 382 and the like will be suitably omitted. Incidentally, the reflection state changing function 382 according to the present application example can be executed along with the reflection state changing function 382 according to another application example and at least one function of other various state changing functions.

(Reflection State Changing Function)

The system control circuitry 38 controls the supply of power to the light source 674 such that the light source 674 is turned ON when the attachment and detachment detector 73 detects that the reflection unit 68 is attached to the support arm 65, and the rotation detector 74 detects the first angle as the positioning state of the reflection unit 68. Incidentally, the system control circuitry 38 may turn ON the light source 674 in response to the detection of the attachment of the reflection unit 68 to the support arm 65 when the attachment and detachment of the reflection unit 68 to and from the support arm 65 can be executed at the first angle. The other control with respect to the light source 674 is duplicated with the above-described content, and thus, will not be described.

According to the above-described configuration, it is possible to obtain effects to be described as follows in addition to the above-described effects.

According to the magnetic resonance diagnostic apparatus 10 according to the first application example, it is possible to switch the ON state and the OFF state of the light source 674 or the back monitor 676 mounted to the reflection unit 68 in response to the attachment and detachment of the reflection unit 68 to and from the support arm 65. Accordingly, it is possible to set the back of the beam splitter 671 to be brighter than the front of the beam splitter 671 in the reflection unit 68 attached to the support arm 65 at the first angle.

Accordingly, the face of the patient P is not practically reflected on the reflection unit 68 attached to the support arm 65 at the first angle, that is, in substantially horizontal state even when the reflection unit 68 is detachable from the support arm 65, the patient P can visually recognize the image of the film 672 arranged at the back of the beam splitter 671 or the image of the back monitor 676 directly instead of visually recognizing the face through the reflection unit 68 according to the magnetic resonance diagnostic apparatus 10 according to the first application example. Accordingly, it is possible to provide environment in which the sense of discomfort of the patient P can be reduced, and the patient P can relax.

(Second Application Example)

The second application example corresponds to a case in which the detector 72 includes the rotation detector 74 and the movement detector 75. The system control circuitry 38 that realizes the reflection state changing function 382 controls the supply of power to the light source 674 according to at least one of the relative position of the reflection unit 68 with respect to the screen 63 and the relative position of the support arm 65 with respect to the screen 63 in order to switch the ON state and the OFF state of the light source 674.

To be specific, the system control circuitry 38 controls the supply of power to the light source 674 according to the movement of the support arm 65 and the angle of the reflection unit 68 with respect to the screen 63 in order to switch the ON state and the OFF state of the light source 674.

In the following description, a description regarding redundant parts of the above description on the reflection state changing function 382 will be suitably omitted. Incidentally, the reflection state changing function 382 according to the present application example can be executed along with the reflection state changing function 382 according to another application example and at least one function of other various state changing functions.

Hereinafter, it is assumed that an initial state of the movable screen apparatus 15 is the first positioning mode, and the angle of the reflection unit 68 is the first angle in order to specify the description. In addition, it is assumed that the support arm 65 is slid to the screen 63 side at this time.

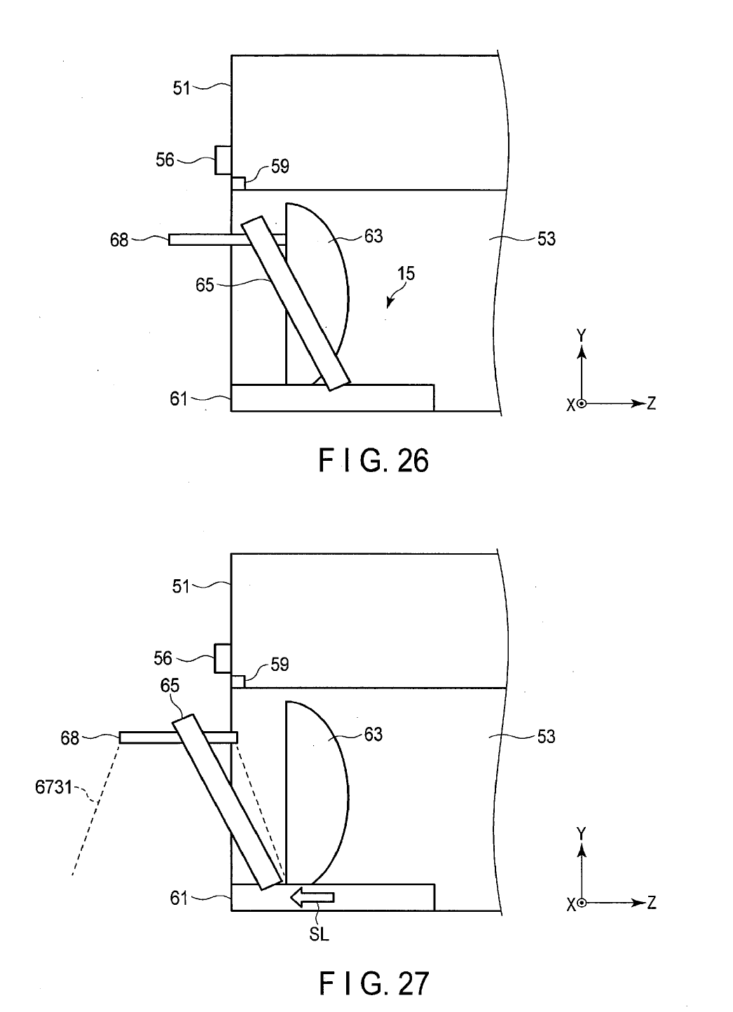

FIG. 26 is a diagram illustrating an example of the movable screen apparatus 15 (in the initial state) which includes the reflection unit 68 arranged at the first angle in the first positioning mode. As illustrated in FIG. 26, the movable screen apparatus 15 is arranged at the end on the couch 13 side. As illustrated in FIG. 26, the support arm 65 is arranged on the screen 63 side. As illustrated in FIG. 26, the reflection unit 68 is arranged at the first angle.

When the movement detector 75 detects the movement of the support arm 65 to the couch 13 side, the system control circuitry 38 supplies current to the light source 674 such that the light source 674 is turned ON. That is, when the relative position of the support arm 65 with respect to the screen 63 is changed from the screen 63 side to the couch 13 side, the light source 674 is turned ON. In addition, when the angle of the reflection unit 68 is different from the first angle in the first positioning mode, the system control circuitry 38 supplies current to the light source 674 in response to both the detection of the movement of the support arm 65 to the couch 13 side and the detection of the first angle.

FIG. 27 is a diagram illustrating an example of the movable screen apparatus 15 at a time at which the support arm 65 is slid to the couch 13 side. As illustrated in FIG. 27, the light source 674 is turned ON in response to movement (slide) SL of the support arm 65 from the screen 63 side to the couch 13 side. At this time, the patient P placed on the couch top 131 can visually recognize the image of the film 672.

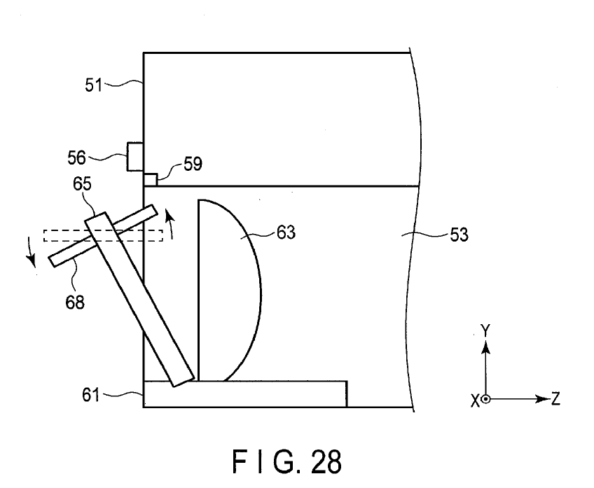

The system control circuitry 38 controls the supply of power to the light source 674 to turn OFF the light source 674 being turned ON in response to the detection of rotation of the reflection unit 68 from the first angle to the second angle. That is, the reflection unit 68, which has been arranged at the first angle with the light source 674 being turned ON, is rotated to the second angle, the supply of current to the light source 674 is cut off, and the light source 674 is turned OFF. FIG. 28 is a diagram illustrating an example of the movable screen apparatus 15 in a state in which the supply of current to the light source 674 is cut off. As illustrated in FIG. 28, the light source 674 is turned OFF in response to the rotation of the reflection unit 68 from the first angle to the second angle.

After the magnetic resonance imaging with respect to the patient P ends or the like, the system control circuitry 38 controls the supply of power to the light source 674 in order to turn ON the light source 674 being turned OFF in response to the detection of rotation of the reflection unit 68 from the second angle to the first angle. That is, the reflection unit 68, which has been arranged at the second angle with the light source 674 being turned OFF, is rotated to the first angle, current is supplied to the light source 674, and the light source 674 is turned ON. FIG. 27 corresponds to the diagram at this time.

When the movement detector 75 detects the movement of the support arm 65 to the screen 63 side, the system control circuitry 38 controls the supply of power to the light source 674 such that the light source 674 is turned OFF. That is, when the relative position of the support arm 65 with respect to the screen 63 is changed from the couch 13 side to the screen 63 side, the supply of current to the light source 674 is cut off, and the light source 674 is turned OFF. FIG. 26 corresponds to the diagram at this time.

(Reflection State Changing Function)

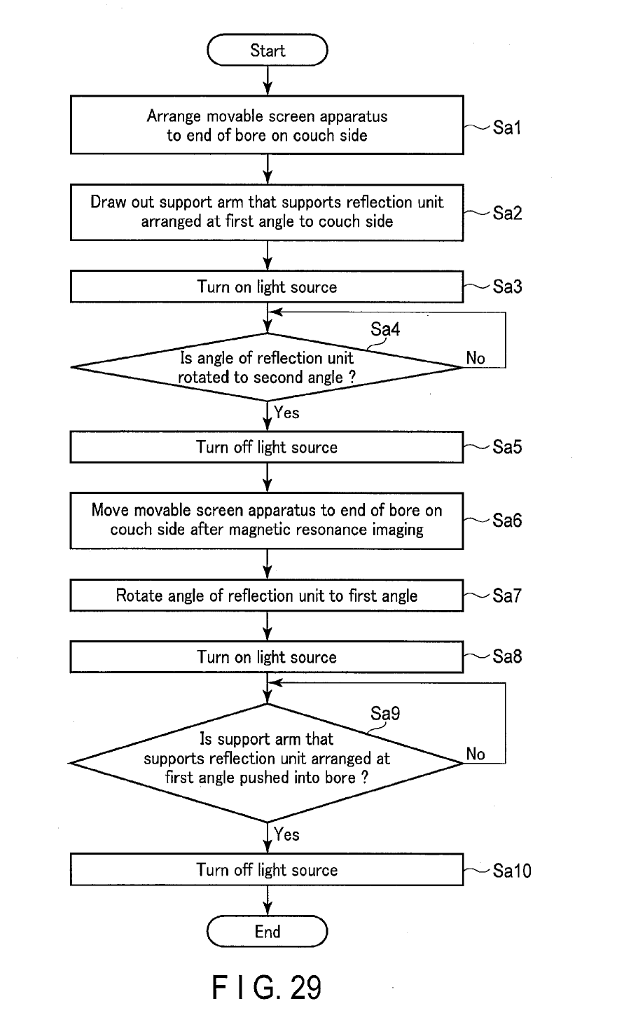

FIG. 29 is a flowchart illustrating an example of procedure of a process relating to the reflection state changing function 382 according to the second application example.

The movable screen apparatus 15 is arranged at the end of the bore 53 on the couch 13 side by a user such as a medical professional before the patient P enters the examination room (Step Sa1). At this time, the reflection unit 68 is arranged at the first angle (to be substantially horizontal). Incidentally, the first angle of the reflection unit 68 is not limited to be substantially horizontal, but may be set to an arbitrary angle according to the physique of the patient P or the like.

A state of the movable screen apparatus 15 in Step Sa1 becomes the first positioning mode. At this time, the patient P can visually recognize the image PI projected on the screen 63 without the intervention of the reflection unit 68 from the outside of the gantry housing 51 as illustrated in FIG. 15. That is, it is possible to prevent the bore 53 from entering the field of view when the patient P views the gantry 11 from the outside of the gantry 11 according to the first positioning mode in which the movable screen apparatus 15 is arranged at a couch-side end PE1 of the bore 53.

As illustrated in FIG. 16, the patient P enters the examination room 300 when the image from the projector 100 is projected on the screen 63 in the first positioning mode. Since the image PI is projected on the screen 63 at this time, the bore 53 blunts the visual recognition of the patient P on being present inside the examination space, and it is possible to relieve the sense of fear against entering the bore 53.

The patient P who has visually recognized the image projected on the movable screen apparatus 15 is placed on the couch top 131. Next, the couch top 131 moves upward in the vertical direction, and is coupled with the movable screen apparatus 15. To be specific, the user presses a lift button provided in the gantry 11 or the couch 13. When receiving the press of the lift button, the imaging control circuitry 31 supplies an electrical signal (hereinafter, referred to as a lift signal), which corresponds to lift of the couch top 131, to the couch driving device 135. When receiving the lift signal, the couch driving device 135 lifts the couch top 131 in the Y-axis direction.

The support arm 65, which supports the reflection unit 68 arranged at the first angle, is drawn out to the couch 13 side by the user's operation after the couch top 131 and the movable screen apparatus 15 are coupled with each other (Step Sa2). At this time, the movement of the support arm 65 on the movable carriage 61 or the position of the support arm 65 having moved to the couch 13 side on the movable carriage 61 is detected by the movement detector 75.

The switching of the light source 674 is controlled and current is supplied to the light source 674 in response to the detection of the movement of the support arm 65 or the position on the couch 13 side. Accordingly, the light source 674 is turned ON (Step Sa3). The ON state of the light source 674 is maintained until the angle of the reflection unit 68 is rotated to the second angle (No in Step Sa4).

When the angle of the reflection unit 68 is rotated from the first angle to the second angle by the user's operation (Yes in Step Sa4), the second angle is detected by the rotation detector 74. The switching of the light source 674 is controlled, and the supply of current to the light source 674 is cut off in response to the detection of the second angle. Accordingly, the light source 674 is turned OFF (Step Sa5).

The user presses an insertion button provided in the gantry 11 or the couch 13. When receiving the press of the insertion button, the imaging control circuitry 31 supplies an electrical signal (hereinafter, referred to as an insertion signal), which corresponds to insertion of the couch top 131, to the couch driving device 135. When receiving the insertion signal, the couch driving device 135 slides the couch top 131 in the positive Z-axis direction. Since the couch top 131 and the movable screen apparatus 15 are coupled with each other, the movable screen apparatus 15 also slides in the positive Z direction while being interlocked with the slide of the couch top 131.

The insertion button is pressed until a part as a photographing target is positioned immediately below the projector 59. When the part as the photographing target is moved immediately below the projector 59, for example, at a position irradiated with the intersection line light 591, the user end the press of the insertion button to stop the couch top 131. Next, the user presses a positioning button provided in the gantry 11 or the couch 13. At this time, the imaging control circuitry 31 temporarily stores the position of the couch top 131 immediately below the projector 59 as a couch top position. When the positioning instruction is input via the input circuitry 36 or the like, the imaging control circuitry 31 controls the couch driving device 135 such that, the couch top position is aligned at the center position of the magnetic field. At this time, the part as the photographing target is moved to the center position of the magnetic field.

FIG. 17 is a diagram illustrating the movable screen apparatus 15 in the second projection format from a lateral side of the gantry 11. As illustrated in FIG. 17, the patient P placed on the couch top 131 can view the image projected on the front surface of the screen 63 via the reflection unit 68 in the second projection format. Since the couch top 131 and the movable screen apparatus 15 are coupled with each other, the distance between the patient P and the screen 63 is kept constant regardless of the slide of the movable screen apparatus 15 in the Z-axis direction. Accordingly, it is possible to enhance the sense of immersion into the image projected on the screen 63 and to relieve the sense of blockage inside the bore 53. The projection of the image according to the second projection format is continued from Step Sa5 to the end of Step Sa7.

When the part as the photographing target is moved to the center position of the magnetic field, the MR imaging is performed. To be specific, the user presses a start button of MR imaging in the control room 400. When the start button is pressed, the imaging control circuitry 31 controls the gradient power supply 21, the transmitting circuitry 23, and the receiving circuitry 25 in a synchronous manner according to an imaging sequence set in advance, and performs the MR imaging. The MR signal relating to the patient P is collected by the receiving circuitry 25 through the MR imaging, and the MR image is reconstructed based on the MR signal by the reconstruction circuitry 32. The patient P can view the image projected on the screen 63 through the reflection unit 68 during the MR imaging. Accordingly, the patient P can comfortably stay inside the bore 53 during the MR imaging taking a relatively long period of time.

When the MR imaging is ended, the couch top 131 is retreated outside the bore 53. That is, the movable screen apparatus 15 is moved to the end of the bore 53 on the couch 13 side when the magnetic resonance imaging with respect to the patient is ended (Step Sa6).

To be specific, the user presses a retreat button provided in the gantry 11 or the couch 13. When receiving the press of the retreat button, the imaging control circuitry 31 supplies an electrical signal (hereinafter, referred to as a retreat signal), which corresponds to retreat of the couch top 131, to the couch driving device 135. When receiving the retreat signal, the couch driving device 135 slides the couch top 131 in the negative Z-axis direction. When the couch top 131 is slid to the outside of the gantry 11, the movable screen apparatus 15 is arranged at the couch-side end PE1 of the bore 53. The patient P can continuously view the image projected on the screen 63 through the reflection unit 68 while the couch top 131 is being moved outside the bore 53.

When the angle of the reflection unit 68 is rotated from the second angle to the first angle by the user's operation (Step Sa7), the first angle is detected by the rotation detector 74. The switching of the light source 674 is controlled, and current is supplied to the light source 674 in response to the detection of the first angle. Accordingly, the light source 674 is turned ON (Step Sa8). The ON state of the light source 674 is maintained until the support arm 65, which supports the reflection unit 68 arranged at the first angle, is pushed into the bore 53, that is, to the screen 63 side (No in Step Sa9).

The movement of the support arm 65 on the movable carriage 61 or the position of the support arm 65 moved to the screen 63 side on the movable carriage 61 is detected by the movement detector 75 when the support arm 65 is pushed into the bore 53, that is, to the screen 63 side by the user's operation (Yes in Step Sa9). The switching of the light source 674 is controlled, and the supply of current to the light source 674 is cut off in response to the detection of the movement of the support arm 65 or the position on the screen 63 side. Accordingly, the light source 674 is turned OFF (Step Sa10).

The couch top 131 is lowered when Step Sa10 is performed, and the coupling between the couch top 131 and the movable screen apparatus 15 is released. To be specific, the user presses a lowering button provided in the gantry 11 or the couch 13. When receiving the press of the lowering button, the imaging control circuitry 31 supplies an electrical signal (hereinafter, referred to as a lowering signal), which corresponds to lowering of the couch top 131, to the couch driving device 135. When receiving the lowering signal, the couch driving device 135 lowers the couch top 131 in the Y-axis direction. When the couch top 131 is lowered, the coupling between the couch top 131 and the movable screen apparatus 15 is released. When the couch top 131 is lowered to an initial position, the user ends the press of the lowering button. Thereafter, the patient P takes off the couch top 131, and exits the examination room 300.

Incidentally, the above-described flow of the MR examination is illustrative, and operation examples of the magnetic resonance imaging system according to the embodiment 1 are not limited to the above-described flow. For example, the lift button and the insertion button are individually pressed to move the couch top 131 from the initial position to a photographing position inside the bore 53 in the above-described flow.

However, the present embodiment is not limited thereto. For example, an automatic insertion button that collectively instructs the lift and insertion of the couch top 131, may be pressed. In addition, the retreat button and the lowering button are individually pressed in order to move the couch top 131 from the inside of the bore 53 to the initial position in the above-described flow. However, the present embodiment is not limited thereto. For example, an automatic retreat button that collectively instructs the retreat and lowering of the couch top 131, may be pressed.

According to the above-described configuration, it is possible to obtain effects to be described as follows in addition to the above-described effects.

According to the magnetic resonance diagnostic apparatus 10 according to the second application example, it is possible to switch the ON state and the OFF state of the light source 674 or the back monitor 676 mounted to the reflection unit 68 in order to switch the ON state of the light source 674 and the OFF state in response to the movement of the support arm 65 and the angle of the reflection unit 68 with respect to the screen 63. Accordingly, it is possible to turn the light source 674 and the back monitor 676 into the OFF state when the support arm 65 is arranged on the screen side on the movable carriage 61, and it is possible to reduce power consumption relating to the light source 674 and the back monitor 676. In this manner, it is possible to improve an environment protection function according to the magnetic resonance diagnostic apparatus 10 according to the second application example.

(Third Application Example)

A third application example corresponds to a case in which a photographing unit is mounted to an exterior of at least one of the support arm 65 and the reflecting plate 67. The photographing unit photographs a figure on the couch top 131, that is, the patient P placed on the couch top 131. The photographing unit is, for example, an optical camera. In the third application example, the photographing unit is controlled by the system control circuitry 38 that realizes a photographing state changing function 383 to be described later. Incidentally, the photographing state changing function 383 according to the present application example can be executed along with the photographing state changing function 383 according to another application example and at least one function of other various state changing functions.

Hereinafter, a description will be given assuming that two photographing units are mounted to the reflecting plate 67. Incidentally, the number of photographing units to be mounted to the reflecting plate 67 is not limited to two. In addition, a case in which a single photographing unit is mounted to the reflecting plate 67 will be described in a fourth application example.

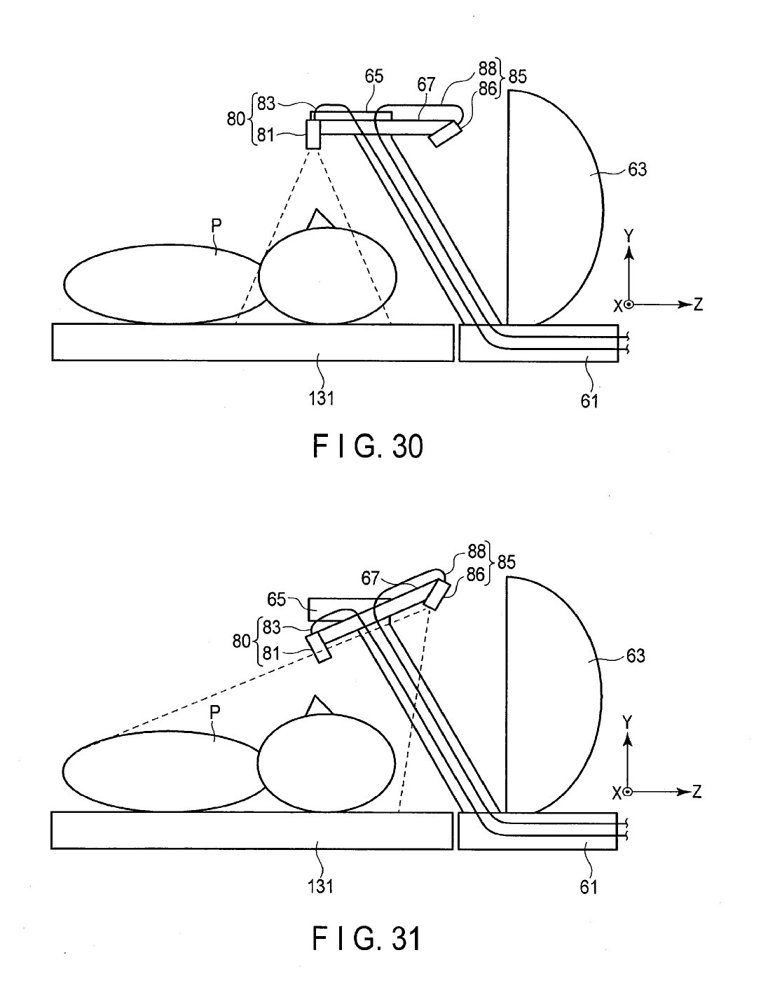

FIG. 30 relates to the third application example of the present embodiment and is a side view of the movable screen apparatus 15 which includes the reflecting plate 67 arranged at the first angle. FIG. 31 relates to the third application example of the present embodiment and is a side view of the movable screen apparatus 15 which includes the reflecting plate 67 arranged at the second angle. As illustrated in FIGS. 30 and 31, two optical cameras 80 and 85, configured to photograph the patient P placed on the couch top 131, are provided at ends on the exterior of the reflecting plate 67 in the movable screen apparatus 15 according to the third application example.

The reflecting plate 67 supports the two photographing unit. To be specific, a photographing direction with respect to the reflecting plate 67 is fixed at the exterior of the reflecting plate 67 or the end on the couch 13 side and the end on the screen 63 side in the base cover 675, thereby supporting the optical camera 80 and the optical camera 85.

Hereinafter, the optical camera mounted on the couch 13 side and the optical camera mounted on the screen 63 side in the reflecting plate 67 will be referred to as a first camera 80 and a second camera 85, respectively, in order to simplify the description. As illustrated in FIGS. 30 and 31, the photographing direction of the first camera 80 and the photographing direction of the second camera 85 are different from each other.

For example, the first camera 80 includes a first objective lens 81, a first optical fiber 83, and a first charge-coupled device (CCD) (not illustrated). For example, the second camera 85 includes a second objective lens 86, a second optical fiber 88, and a second CCD (not illustrated). The first objective lens 81, the first optical fiber 83, the second objective lens 86, and the second optical fiber 88 are formed using a non-magnetic material.

The first objective lens 81 is provided at the end of the reflecting plate 67 on the couch 13 side so as to be capable of photographing the face of the patient P placed on the couch top 131 in the reflecting plate 67 arranged at the first angle. Preferably, the first objective lens 81 is provided at a position opposing the face of the patient P in the reflecting plate 67 arranged at the first angle. A dotted line in FIG. 30 indicates a field of view (photographing range) of the first objective lens 81. As illustrated in FIG. 30, the field of view of the first objective lens 81 is a range that includes the head including the face of the patient P.

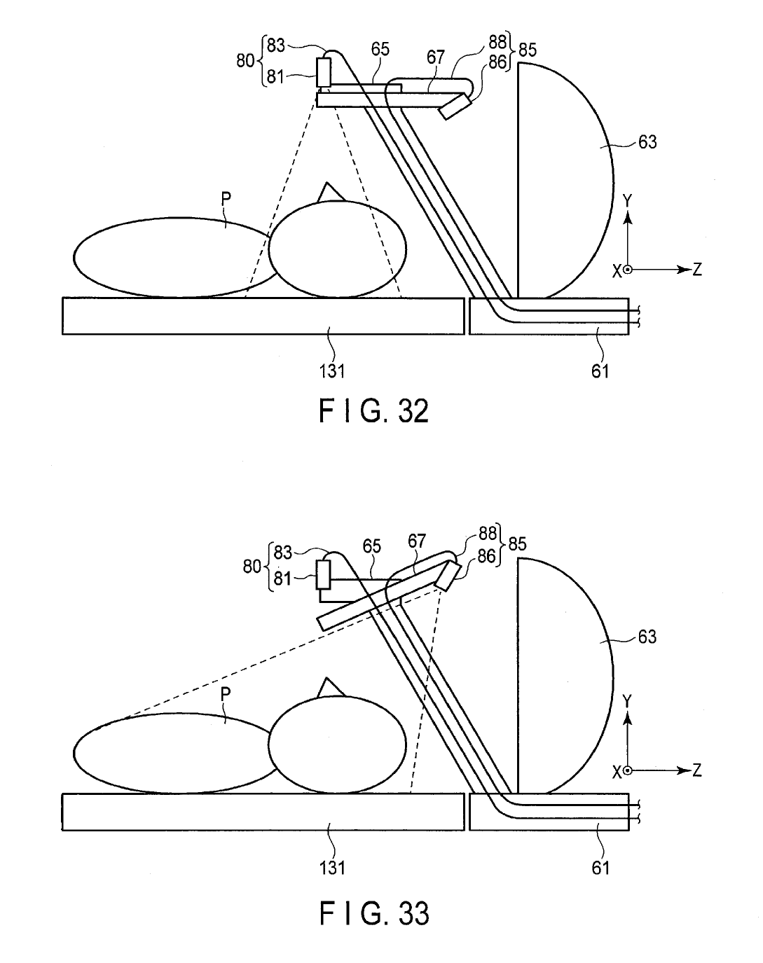

Incidentally, the first objective lens 81 may be provided in the support arm 65 as illustrated in FIG. 32. At this time, as illustrated in FIG. 33, the second objective lens 86 is provided at the end of the reflecting plate 67 on the screen 63 side similarly to FIG. 31. In addition, the first objective lens 81 may be attached in any direction as long as the range that includes the head including the face of the patient P can be photographed.

The second objective lens 86 is provided at the end of the reflecting plate 67 on the screen 63 side so as to be capable of photographing substantially an upper body from the head to the chest or abdomen of the patient P placed on the couch top 131 in the reflecting plate 67 arranged at the second angle. That is, the photographing direction of the second objective lens in the reflecting plate 67 arranged at the second angle is a direction that photographs the patient P in a wide range as much as possible. A dotted line in FIG. 31 indicates a field of view of the second objective lens 86. As illustrated in FIG. 31, the field of view (photographing range) of the second objective lens 86 is a range that includes substantially the upper body of the patient P. The first objective lens 81 and the second objective lens 86 converge or diverge light from the patient P.

The first optical fiber 83 is an optical waveguide which guides light from the first objective lens 81. The first optical fiber 83 connects the first objective lens 81 and the first CCD so as to guide the light from the first objective lens 81 to the first CCD provided at the outside of the gantry 11.

The second optical fiber 88 is an optical waveguide that guides light from the second objective lens 86. The second optical fiber 88 connects the second objective lens 86 and the second CCD so as to guide the light from the second objective lens 86 to the second CCD provided at the outside of the gantry 11.

In addition, the first optical fiber 83 and the second optical fiber 88 may be attached to the rail 55 or a front surface of the inner wall 57 of the gantry housing 51, or may be embedded inside the rail 55 or the gantry housing 51.

The first CCD includes a plurality of light receiving elements that receive the light from the first optical fiber 83 and convert the light into electrical signals. The second CCD includes a plurality of light receiving elements that receive the light from the second optical fiber 88 and convert the light into electrical signals. The first CCD and the second CCD generate optical image data relating to the patient P based on the electrical signals from the plurality of light receiving elements. For example, the optical image data generated by the first CCD is data (hereinafter, referred to as facial image data) mainly relating to a facial image of the patient P as illustrated in FIGS. 30 and 32. In addition, the optical image data generated by the second CCD is data (hereinafter, referred to as upper body image data) mainly relating to an upper body image of the patient P as illustrated in FIGS. 31 and 33.

Incidentally, the first optical fiber 83 and the second optical fiber 88 are illustrated as different optical fibers in FIGS. 30 to 33, but may be provided as a single optical fiber in the support arm 65, for example. At this time, a single CCD is provided on the outside of the gantry 11. In addition, the first optical fiber 83 and the second optical fiber 88 are connected to the exterior of the support arm 65 or the reflecting plate 67, a switcher (not illustrated) is mounted to switch the light from the first objective lens 81 and the light from the second objective lens 86. Incidentally, the switcher may be provided near the single CCD provided on the outside of the gantry 11.

Incidentally, the first optical fiber 83 that connects the first objective lens 81 and the CCD and the second optical fiber 88 that connects the second objective lens 86 and the CCD have rigidity. Thus, there is a risk that the first optical fiber 83 and the second optical fiber 88 are damaged along with the slide of the movable screen apparatus 15. In order to prevent such a risk, for example, a mechanism, which is capable of connecting the first objective lens 81 and the second optical fiber 88 with the CCD while maintaining a constant curvature of the first optical fiber 83 and the second optical fiber 88 regardless of the slide of the movable screen apparatus 15, may be provided in the gantry 11 or the like.

The optical image data is supplied to the console 27 via the communication circuitry 34. An optical image corresponding to the supplied optical image data is displayed by the display circuitry 35. A medical professional or the like can monitor the patient P during the MR imaging by observing the optical image. To be specific, the optical image (hereinafter, referred to as the facial image) corresponding to the facial image data is displayed on various displays in the console 27 and the touch panel on the exterior surface of the gantry housing 51 on the couch 13 side.

In addition, the optical image (hereinafter, referred to as the upper body image) corresponding to the upper body image data is displayed on the various displays in the console 27. Incidentally, the upper body image may be displayed on the touch panel on the exterior surface of the gantry housing 51. The upper body image includes position information (body movement information) as a feature point that changes depending on body movement of the patient P, which will be described later.

The input circuitry 36 inputs the positioning instruction with respect to the facial image displayed on the touch panel provided on the exterior surface of the gantry housing 51 on the couch 13 side. Accordingly, the couch top position is set with respect to the head region of the patient P.

The image processing circuitry 33 performs body movement correction of a reconstructed image using the upper body image photographed by the second camera 85. For example, the image processing circuitry 33 tracks positions of the feature point of time-series upper body images in time series, and calculates the movement amount of the feature point. The feature point can be set to an arbitrary part (anatomic feature point) such as the chin or forehead of the patient P, the cervical spine, and a marker attached to the patient P which is depicted on the upper body image. Then, the image processing circuitry 33 generates an image in which the body movement is corrected by performing coordinate conversion of the reconstructed image according to the calculated movement amount.

(Photographing State Changing Function)

The photographing state changing function 383 is a function of changing a photographing state to optically photograph the inside of the bore 53 according to at least one of the angle of the reflecting plate 67 with respect to the screen 63 and the relative position of the support arm 65 with respect to the screen 63. Hereinafter, a description will be given regarding a process according to the photographing state changing function 383.

The system control circuitry 38 that realizes the photographing state changing function 383 switches the photographing direction of the photographing unit to change the photographing state based on the detection result from the detector 72. The switching of the photographing direction is realized by, for example, switching between an ON state (hereinafter, referred to as a photographing ON state) and an OFF state (hereinafter, referred to as a photographing OFF state) of photographing performed by the first camera 80 and the second camera 85. The switching between the photographing ON state and the photographing OFF state is performed according to the angle of the reflecting plate 67 under the control performed by the system control circuitry 38.

The switching between the photographing ON state and the photographing OFF state corresponds to switching between an image generable state and an image ungenerable state of the CCD. At this time, a control target according to the system control circuitry 38 is the first CCD and the second CCD. Incidentally, when the single optical fiber is connected to the CCD, the system control circuitry 38 controls the switcher according to the angle of the reflecting plate 67. Any light between the light from the first objective lens 81 and the light from the second objective lens 86 is guided to the CCD by the control of the switcher.

To be specific, the system control circuitry 38 controls the first CCD such that the first CCD is maintained in the photographing ON state and controls the second CCD such that the second CCD is maintained in the photographing OFF state when the first angle is detected by the detector 72, or the support arm 65 is positioned on the screen 63 side on the movable carriage 61. At this time, the system control circuitry 38 controls the switcher such that the light from the first objective lens 81 is guided to the CCD when the single optical fiber is connected to the CCD.

A period in which the first CCD is in the photographing ON state and the second CCD is in the photographing OFF state, or a period in which the light from the first objective lens 81 is guided to the CCD corresponds to, for example, a period from Step Sa1 to No in Step Sa4 and a period from Step Sa8 to the end of the flowchart in FIG. 29.

The system control circuitry 38 controls the first CCD such that the first CCD is maintained in the photographing OFF state and controls the second CCD such that the second CCD is maintained in the photographing ON state when the second angle is detected by the detector 72 or when the support arm 65 is positioned on the couch 13 side on the movable carriage 61. At this time, the system control circuitry 38 controls the switcher such that the light from the second objective lens 86 is guided to the CCD when the single optical fiber is connected to the CCD.

A period in which the first CCD is in the photographing OFF state and the second CCD is in the photographing ON state, or a period in which the light from the second objective lens 86 is guided to the CCD corresponds to, for example, a period from Yes in Step Sa4 to Step Sa7 of the flowchart in FIG. 29.

In addition, the system control circuitry 38 controls the first CCD such that the photographing state of the first CCD is changed from the photographing ON state to the photographing OFF state and controls the second CCD such that the photographing state of the second CCD is changed from the photographing OFF state to the photographing ON state when the positioning state of the reflecting plate 67 is changed from the first angle to the second angle, that is, when the start time of the rotating operation of the reflecting plate 67 about the rotation shaft RR1 is detected. At this time, the switcher is controlled such that the light from the first objective lens 81 is cut off and the light from the second objective lens 86 is guided to the CCD when the single optical fiber is connected to the CCD. The process of this time corresponds to, for example, Step Sa5 of the flowchart in FIG. 29.

In addition, the system control circuitry 38 controls the first CCD such that the photographing state of the first CCD is changed from the photographing OFF state to the photographing ON state and controls the second CCD such that the photographing state of the second CCD is changed from the photographing ON state to the photographing OFF state when the positioning state of the reflecting plate 67 is changed from the second angle to the first angle. At this time, the system control circuitry 38 controls the switcher such that the light from the second objective lens 86 is cut off and the light from the first objective lens 81 is guided to the CCD when the single optical fiber is connected to the CCD. The process of this time corresponds to, for example, Step Sa8 of the flowchart in FIG. 29.

Incidentally, the system control circuitry 38 may control the first CCD such that the photographing state of the first CCD is switched from the photographing OFF state to the photographing ON state when the support arm 65 is drawn out from the screen 63 side to the couch 13 side on the movable carriage 61. At this time, the system control circuitry 38 controls the switcher such that the light from the first objective lens 81 is guided to the CCD when the single optical fiber is connected to the CCD.

In addition, the system control circuitry 38 may control the first CCD and the second CCD such that both the photographing states of the first CCD and the second CCD are turned into the photographing OFF state when the support arm 65 is pushed from the couch 13 side to the screen 63 side on the movable carriage 61.

FIGS. 30 and 32 are diagrams illustrating the side face of the movable screen apparatus 15 when the reflecting plate 67 is arranged at the first angle. As illustrated in FIGS. 30 and 32, the first camera 80 optically photographs the head region of the patient P. At this time, the second camera 85 does not photograph the patient P.

FIGS. 31 and 33 are diagrams illustrating the side face of the movable screen apparatus 15 when the reflecting plate 67 is arranged at the second angle. As illustrated in FIGS. 31 and 33, the second camera 85 optically photographs substantially the upper body of the patient P. At this time, the first camera 80 does not photograph the patient P.

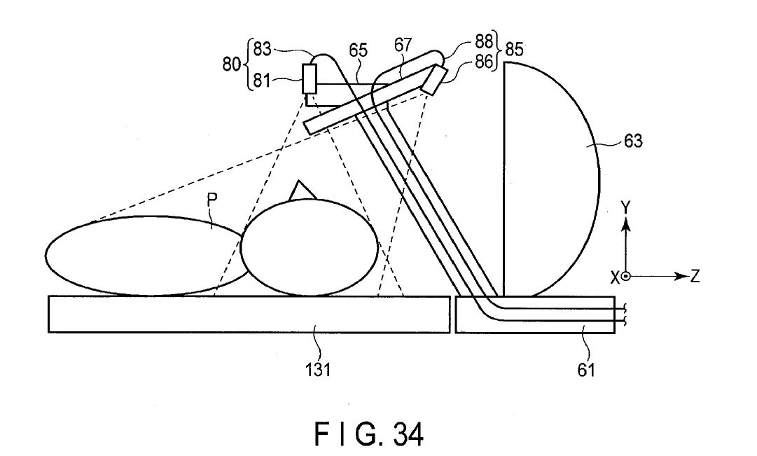

Incidentally, the first camera 80 can photograph the patient P when the first camera 80 is mounted to the support arm 65 and the reflecting plate 67 is arranged at the second angle. That is, the first camera 80 and the second camera 85 can simultaneously photograph the patient P when the reflecting plate 67 is arranged at the second angle. FIG. 34 is a diagram illustrating a field of view of the first camera 80 and a field of view of the second camera 85 when the first camera 80 is mounted to the support arm 65 and the reflecting plate 67 is arranged at the second angle. As illustrated in FIG. 34, the first camera 80 constantly photographs the patient P.

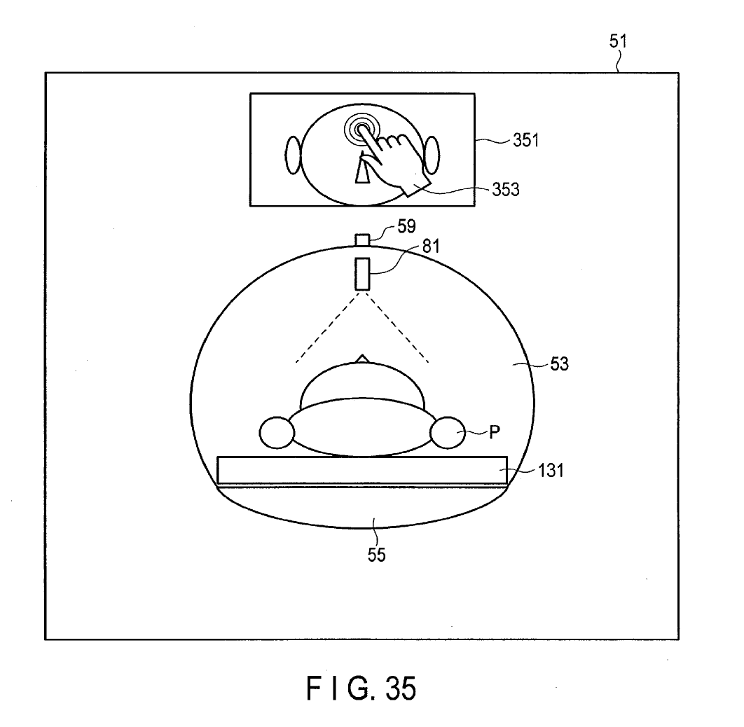

FIG. 35 is a front view when a touch panel 351 on which the facial image of the patient P in FIG. 21 is displayed is viewed from the couch 13 side of the gantry 11. As illustrated in FIG. 35, the facial image of the patient P is displayed on the touch panel 351. The user can input the positioning instruction through a touch operation 353 with respect to the facial image displayed on the touch panel 351.

At this time, the imaging control circuitry 31 stores a position immediately below the first camera 80 as the couch top position that needs to be moved to the center position of the magnetic field. At this time, the imaging control circuitry 31 functions as a setting unit that sets a position, designated by the user, in the vide on the touch panel (video display monitor) as the position that needs to be moved to the center position of the magnetic field. Incidentally, the function as the setting unit may be executed by the system control circuitry 38.

Next, the imaging control circuitry 31 controls the couch driving device 135 such that the couch top position is moved to the center position of the magnetic field when the angle of the reflecting plate 67 is changed from the first angle to the second angle by the user's operation. Accordingly, the part as the photographing target desired by the user is moved to the center position of the magnetic field in the bore 53.

According to the above-described configuration, it is possible to obtain effects to be described as follows.

According to the magnetic resonance diagnostic apparatus 10 that realizes the above-described photographing state changing function 383, it is possible to switch the photographing states using the two cameras according to at least one of the angle of the reflecting plate 67 with respect to the screen 63 and the relative position of the support arm 65 with respect to the screen 63. It is possible to change the photographing direction (direction of the objective lens) and an angle of view with respect to the patient P, for example, by selectively use the camera in response to the angle of the reflecting plate 67 with respect to the screen according to the magnetic resonance diagnostic apparatus 10 according to the third application example of the present embodiment.

Accordingly, the facial image collected by the first camera 80 can be displayed on the touch panel 351, and the positioning instruction can be input with respect to the displayed facial image when the reflecting plate 67 is set at the first angle to be substantially horizontal. That is, the first camera 80 according to the third application example can serves not only a function as the projector 59 but also a function as a monitor of the patient P placed on the couch top 131. At this time, the projector 59 is not required, and it is possible to reduce manufacturing cost of the magnetic resonance diagnostic apparatus 10.

In this manner, when the part as the photographing target is the head region of the patient P, it is possible to perform the positioning of the part as the photographing target to the center position of the magnetic field without using the projector 59, that is, without inserting any part of the patient P into the bore 53 during the positioning with respect to the head of the patient P according to the magnetic resonance diagnostic apparatus 10 according to the third application example. Accordingly, it is unnecessary to move the patient's head immediately below the projector 59 in the case of photographing the patient's head, and thus, the patient P can visually recognize the image of the film 672 arranged at the back of the beam splitter 671 or the image of the back monitor 676 directly even during the positioning according to the magnetic resonance diagnostic apparatus 10 according to the third application example.

In the above-described manner, it is possible to provide the environment in which the reflecting plate 67 arranged at the first angle is effective utilized, the anxiety of the patient P is improved without causing the uncomfortable feeling to the patient P, and the patient P can relax by allowing the patient P to visually recognize the image of the film 672 arranged at the back of the beam splitter 671 or the image of the back monitor 676 in the reflecting plate 67 arranged at the first angle according to the magnetic resonance diagnostic apparatus 10 according to the third application example.

In addition, it is possible to collect the upper body image of the patient P using the second camera 85 when the reflecting plate 67 is set at the second angle (gradient) according to the magnetic resonance diagnostic apparatus 10 according to the third application example. Accordingly, it is possible to monitor the patient P in the wide range when the reflecting plate 67 is set at the second angle according to the magnetic resonance diagnostic apparatus 10 according to the third application example. Further, it is possible to execute the body movement correction of the reconstructed image using the upper body image and to improve the image quality of the reconstructed image or the like according to the magnetic resonance diagnostic apparatus 10 according to the third application example.

Further, the first camera 80 according to the third application example is provided in the movable screen apparatus 15 arranged inside the bore 53, and thus, is provided at a position closer to the patient P than a conventional camera for monitor which is provided outside the gantry 11. Accordingly, it is possible to photograph facial expression of the patient P inside the gantry 11, for example, and thus, it is possible to accurately grasp a state of the patient P during the MR imaging via the monitor provided near the display circuitry 35 in the console 27 or the console 27. In addition, it is possible to photograph not only the face of the patient P but also substantially the upper body of the patient P even when the screen 63 is arranged inside the bore 53, and to monitor the patient P in a preferable state according to the present application example.

(Fourth Application Example)

The fourth application example corresponds to a case in which a photographing unit whose photographing direction can be changed is mounted to an end of the exterior of the reflecting plate 67. The photographing unit is, for example, an optical camera. The photographing direction of the photographing unit is controlled by the system control circuitry 38 that realizes the photographing state changing function 383. In the following description, the description will be suitably omitted regarding redundant parts with the third application example. Incidentally, the photographing state changing function 383 according to the present application example can be executed along with the photographing state changing function 383 according to the third application example and at least one function of other various state changing functions.

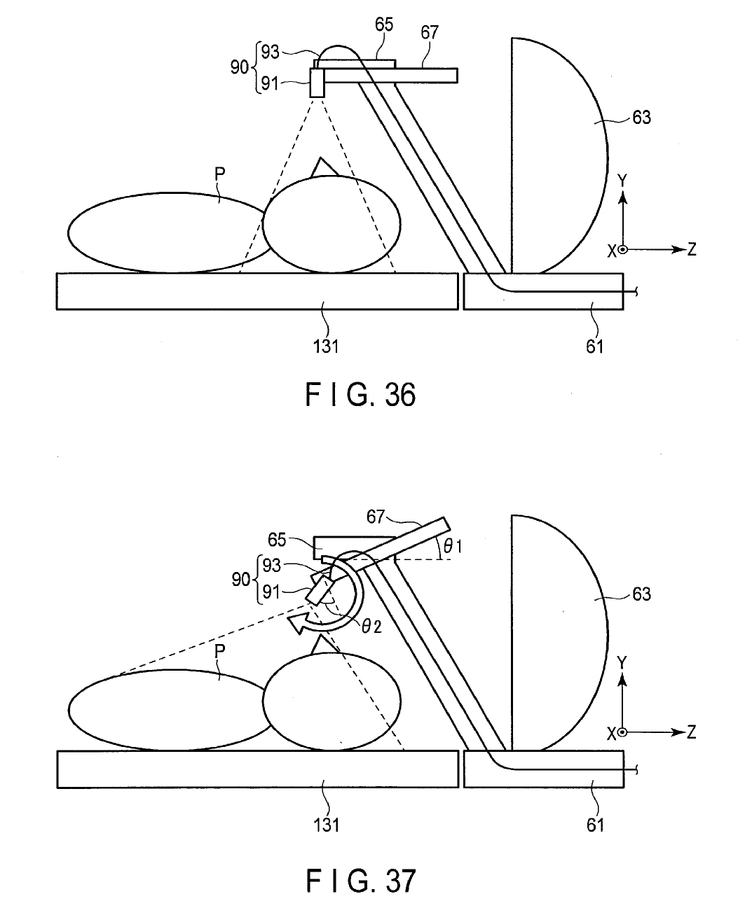

FIG. 36 relates to the fourth application example and is a side view of the movable screen apparatus 15 which includes the reflecting plate 67 arranged at the first angle. FIG. 37 relates to the fourth application example and is a side view of the movable screen apparatus 15 which includes the reflecting plate 67 arranged at the second angle. As illustrated in FIGS. 36 and 37, an optical camera 90, configured to photograph the patient P placed on the couch top 131, is provided at an end on the exterior of the reflecting plate 67 in the movable screen apparatus 15 according to the fourth application example.

The reflecting plate 67 supports a single photographing unit. To be specific, the reflecting plate 67 supports the single optical camera 90 at the end of the exterior of the reflecting plate 67 or the base cover 675 on the couch 13 side such that the photographing direction with respect to the reflecting plate 67 can be changed. For example, the reflecting plate 67 supports an objective lens, which will be described later, via a bearing fit to a rotation shaft (hereinafter, referred to as a lens rotation shaft) that can freely rotate the objective lens in the optical camera 90.

For example, the optical camera 90 includes an objective lens 91, an optical fiber 93, and a CCD (not illustrated). The objective lens 91 and the optical fiber 93 are formed using a non-magnetic material. The objective lens 91 converges or diverges the light from the patient P.

The objective lens 91 is arranged at the end of the reflecting plate 67 on the couch 13 side so as to be directed to oppose the face of the patient P placed on the couch top 131 in the reflecting plate 67 arranged at the first angle. A dotted line in FIG. 36 indicates a field of view (photographing range) of the objective lens 91. As illustrated in FIG. 36, a field of view of the objective lens 91 is a range that includes the head including the face of the patient P. At this time, the objective lens 91 according to the present application example corresponds to the first objective lens 81 of the fifth application example.

The objective lens 91 is arranged at the end of the reflecting plate 67 on the couch 13 side so as to be capable of photographing substantially the upper body from the head to the chest or abdomen of the patient P placed on the couch top 131 in the reflecting plate 67 arranged at the second angle. That is, the photographing direction of the objective lens 91 in the reflecting plate 67 arranged at the second angle is a direction that photographs the patient P in a wide range as much as possible. A dotted line in FIG. 37 indicates the field of view of the objective lens 91. As illustrated in FIG. 37, the field of view (photographing range) of the objective lens 91 is a range that includes substantially the upper body of the patient P. At this time, the objective lens 91 according to the present application example corresponds to the second objective lens 86 of the fifth application example.

The optical fiber 93 is an optical waveguide that guides light from the objective lens 91. The optical fiber 93 connects the objective lens 91 and the CCD so as to guide the light from the objective lens 91 to the CCD provided at the outside of the gantry 11. The optical fiber 93 may be attached to the rail 55 or a front surface of the inner wall 57 of the gantry housing 51, or may be embedded inside the rail 55 or the gantry housing 51.

The CCD includes a plurality of light receiving elements that receive the light from the optical fiber 93 and convert the light into electrical signals. The CCD generates optical image data relating to the patient P based on the electrical signals from the plurality of light receiving elements. For example, the optical image data generated by the CCD is the facial image data mainly relating to a facial image of the patient P as illustrated in FIG. 36 in the reflecting plate 67 arranged at the first angle. In addition, the optical image data generated by the CCD is the upper body image data mainly relating to an upper body image of the patient P as illustrated in FIG. 37 in the reflecting plate 67 arranged at the second angle.

Incidentally, the optical fiber 93 that connects the objective lens 91 and the CCD has rigidity, and thus, there is a risk that the optical fiber 93 is damaged along with the slide of the movable screen apparatus 15. In order to prevent such a risk, for example, a mechanism, which is capable of connecting the objective lens 91 and the CCD while maintaining a constant curvature of the optical fiber 93 regardless of the slide of the movable screen apparatus 15, may be provided in the gantry 11 or the like.

The facial image corresponding to the facial image data is displayed on various displays in the console 27 and the touch panel on the exterior surface of the gantry housing 51 on the couch 13 side. In addition, the upper body image corresponding to the upper body image data is displayed on the various displays in the console 27. Incidentally, the upper body image may be displayed on the touch panel on the exterior surface of the gantry housing 51. The upper body image includes the body movement information that changes depending on the body movement of the patient P.

The input circuitry 36 inputs the positioning instruction with respect to the facial image displayed on the touch panel provided on the exterior surface of the gantry housing 51 on the couch 13 side. Accordingly, the couch top position is set with respect to the head region of the patient P.

The image processing circuitry 33 performs body movement correction of a reconstructed image using the upper body image photographed by the optical camera 90 in the reflecting plate 67 arranged at the second angle.

A rotation transfer mechanism (not illustrated), which transfers the rotation of the rotation shaft RR1 to the lens rotation shaft, is provided between the rotation shaft RR1 of the reflecting plate 67 and the lens rotation shaft. Examples of the rotation transfer mechanism include a flexible shaft, various gears, various transmitting mechanisms (chain and sprocket, pulley and belt, a shaft drive, and the like), and a universal joint. The rotation transfer mechanism is configured using a non-magnetic material.

To be specific, the rotation transfer mechanism is the flexible shaft that connects one end of the rotation shaft RR1 and one end of the lens rotation shaft. Incidentally, the rotation transfer mechanism may be configured of a first gear fit to the rotation shaft RR1, a second gear fit to the lens rotation shaft, a shaft provided with a chain that transfers rotation of the first gear to the second gear or teeth on both ends thereof. In addition, the rotation transfer mechanism may be configured of a first pulley fit to the rotation shaft RR1, a second pulley fit to the lens rotation shaft, and a belt that transfers rotation of the first pulley to the second pulley. In addition, the rotation transfer mechanism may be the universal joint that connects the rotation shaft RR1 and the lens rotation shaft.

In general, a rotation angle θ1 of the reflecting plate 67 about the rotation shaft RR1 and a rotation angle θ2 of the objective lens 91 about the lens rotation shaft are different from each other as illustrated in FIGS. 36 and 37. Thus, the rotation transfer mechanism includes an angle alignment mechanism (not illustrated) that causes the rotation angle θ1 and the rotation angle θ2 to be aligned with each other. The angle alignment mechanism is realized by, for example, various gears configured using a non-magnetic material. Incidentally, the angle alignment mechanism is not required when the rotation angle θ1 and the rotation angle θ2 are equal.

The angle adjusting mechanism, for example, corresponds to at least one gear which is provided at least one of a portion between one end of the rotation shaft RR1 and the flexible shaft and a portion between one end of the lens rotation shaft and the flexible shaft. In addition, when the rotation transfer mechanism is configured of various gears, various transmitting mechanisms, and the universal joint, the angle adjusting mechanism corresponds to the number of teeth of the gear, a length of circumference of the pulley, or the like.

The rotation angle θ1 of the reflecting plate 67 about the rotation shaft RR1 is a difference between the first angle and the second angle. The rotation angle θ2 of the objective lens 91 about the lens rotation shaft is a difference between an angle of the objective lens 91 with respect to the reflecting plate 67 arranged at the first angle and an angle of the objective lens 91 with respect to the reflecting plate 67 arranged at the second angle.

(Photographing State Changing Function)

The rotation transfer mechanism transfers the rotation about the rotation shaft RR1 to the lens rotation shaft when the reflecting plate 67 is rotated from the first angle to the second angle by the user's operation. To be specific, the angle alignment mechanism in the rotation transfer mechanism converts the rotation angle θ1 into the rotation angle θ2. The rotation transfer mechanism transfers the converted rotation angle θ2 to the lens rotation shaft. The lens rotation shaft rotates the objective lens 91 by the rotation angle θ2.

In addition, the rotation transfer mechanism transfers the rotation about the rotation shaft RR1 to the lens rotation shaft when the reflecting plate 67 is rotated from the second angle to the first angle by the user's operation. A difference from the above description is only that a rotation direction is different, and thus, the description thereof will be omitted. The photographing state (photographing direction) of the optical camera 90 is changed through the above-described operation.

According to the above-described configuration, it is possible to obtain effects to be described as follows in addition to the effects described in the third application example.

According to the magnetic resonance diagnostic apparatus 10 that realizes the above-described photographing state changing function 383, it is possible to switch the photographing states using the single optical camera 90 according to the angle of the reflecting plate 67 with respect to the screen 63. That is, it is possible to change the photographing direction of the objective lens 91 with respect to the patient P while being interlocked with the rotation of the reflecting plate 67 with respect to the screen according to the magnetic resonance diagnostic apparatus 10 according to the fourth application example of the present embodiment.

It is possible to change the direction of the objective lens 91 while being interlocked with the rotation of the reflecting plate 67 through the structural mechanism, and thus, it is possible to reduce the manufacturing cost and simplify the movable screen apparatus 15 according to the magnetic resonance diagnostic apparatus 10 according to the fourth application example. In addition, it is possible to photograph not only the face of the patient P but also substantially the upper body of the patient P even when the screen 63 is arranged inside the bore 53, and to monitor the patient P in a preferable state according to the present application example, which is similar to the third application example.

(Fifth Application Example)

A fifth application example corresponds to a case of realizing an illumination state changing function 384 as the state changing function 381. Incidentally, the illumination state changing function 384 according to the present application example can be executed along with the illumination state changing function 384 according to another application example and at least one function of other various state changing functions.

(Illumination State Changing Function)

The illumination state changing function 384 is a function of changing an illumination state of the examination space inside the bore 53 and the examination room 300 according to at least one of the angle of the reflecting plate 67 with respect to the screen 63 and the relative position of the support arm 65 with respect to the screen 63. Hereinafter, a process according to the illumination state changing function 384 will be described.

To be specific, the system control circuitry 38 that realizes the illumination state changing function 384 switches an ON state and an OFF state of illumination or intensity (dimming) of illumination of at least one of the exterior illuminator 56, the in-bore illuminator 58, and the examination room illuminator 310 based on the detection result from the detector 72. The switching between the ON state and the OFF state of the illumination corresponds to, for example, the supply and cut-off of current (switching of current).

In addition, the switching of intensity of the illumination corresponds to, for example, the magnitude of the supply amount of current or a change of the number of LED's to which current is supplied. Incidentally, the amount of light of the illumination may be reduced instead of the OFF state of the illumination. In addition, the system control circuitry 38 may control a width, dimming or the like of light with which the exterior surface on the couch 13 side is irradiated by the exterior illuminator 56 based on the detection result from the detector 72.

To be specific, when the first angle is detected by the detector 72 or when the support arm 65 is positioned on the screen 63 side on the movable carriage 61, the system control circuitry 38 controls at least one of the three illuminators such that the illumination of at least one of the exterior illuminator 56, the in-bore illuminator 58, and the examination room illuminator 310 is maintained in the ON state. A period in which the illumination is maintained in the ON state corresponds to a period from Step Sa1 to No in Step Sa4 and a period from Step Sa8 to the end of the flowchart in FIG. 29, for example.

At this time, the system control circuitry 38 may control the three illuminators sin as to adjust a hue of illumination light and the amount of light of generated by the exterior illuminator 56, the in-bore illuminator 58, and the examination room illuminator 310 according to content of the image projected on the screen 63. For example, when the image as the projection target is an image relating to blue such as the sea and the sky, the system control circuitry 38 controls the three illuminators such that the hue of the illumination light becomes blue. In addition, when the image as the projection target is an image relating to green such as a forest, woods, and a mountain, the system control circuitry 38 controls the three illuminators such that the hue of the illumination light become green.

In addition, the system control circuitry 38 may control the three illuminators according to the amount of light of the image as the image target. For example, when the patient entering the examination room 300 is not allowed to be aware of the image projected on the screen 63 in the first positioning mode, the system control circuitry 38 controls the three illuminators so as to generate illumination light having a larger amount of light than the amount of light of the image as the image target. In addition, when the patient entering the examination room 300 is allowed to be aware of the image projected on the screen 63 in the first positioning mode, the system control circuitry 38 controls the three illuminators so as to generate illumination light having a smaller amount of light than the amount of light of the image as the image target.

When the second angle is detected by the detector 72, or when the support arm 65 is positioned on the couch 13 side on the movable carriage 61, the system control circuitry 38 controls at least one of the three illuminators such that the illumination of the exterior illuminator 56, the in-bore illuminator 58, and the examination room illuminator 310 is maintained in the OFF state. A period in which the illumination is maintained in the OFF state corresponds to a period from Yes in Step Sa4 to Step Sa7 of the flowchart in FIG. 29, for example.

In addition, when the positioning state of the reflecting plate 67 is changed from the first angle to the second angle, that is, when the start time of the rotating operation of the reflecting plate 67 about the rotation shaft RR1 is detected, the system control circuitry 38 controls the three illuminators such that the illumination states of the three illuminators are switched from the ON state to the OFF state. The process of switching the illumination state from the ON state to the OFF state corresponds to Step Sa5 of the flowchart in FIG. 29, for example.

In addition, when the positioning state of the reflecting plate 67 is changed from the second angle to the first angle, the system control circuitry 38 controls the three illuminators such that the illumination states of the three illuminators are switched from the OFF state to the ON state. The process of switching the illumination state from the OFF state to the ON state corresponds to Step Sa8 of the flowchart in FIG. 29, for example.

Incidentally, the system control circuitry 38 may control the three illuminators such that the illumination states of the three illuminators are switched from the ON state to the OFF state when the support arm 65 is drawn out from the screen 63 side to the couch 13 side on the movable carriage 61. In addition, the system control circuitry 38 may control the three illuminators such that the illumination states of the three illuminators are switched from the OFF state to the ON state when the support arm 65 is pushed from the couch 13 side to the screen 63 side on the movable carriage 61.

Incidentally, the switching of the illumination state between the ON state and the OFF state may be executed with respect to at least one illuminator among the exterior illuminator 56, the in-bore illuminator 58, and the examination room illuminator 310.

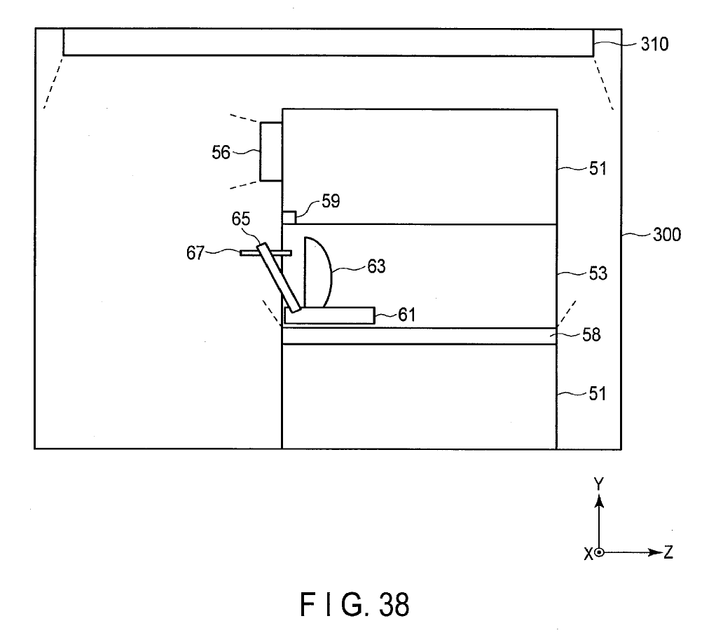

FIG. 38 is a diagram illustrating an example of each illumination state inside the examination room 300 and the bore 53 when the reflecting plate 67 is arranged at the first angle as a positioning state in the first positioning mode. At this time, the illumination of the exterior illuminator 56, the in-bore illuminator 58, and the examination room illuminator 310 is turned into the ON state as illustrated in FIG. 38.

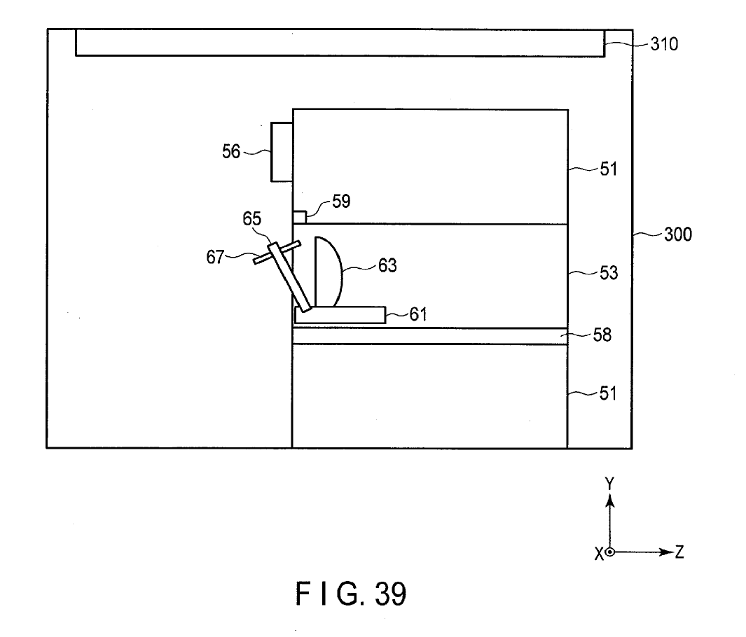

FIG. 39 is a diagram illustrating an example of each illumination state inside the examination room 300 and the bore 53 when the reflecting plate 67 is arranged at the second angle as a positioning state. At this time, the exterior illuminator 56, the in-bore illuminator 58, and the examination room illuminator 310 have the illumination in the OFF state as illustrated in FIG. 39.

According to the above-described configuration, it is possible to obtain effects to be described as follows.

According to the magnetic resonance diagnostic apparatus 10 according to the fifth application example, it is possible to switch the ON state and the OFF state of the illumination or the intensity of the illumination of at least one of the exterior illuminator 56, the in-bore illuminator 58, and the examination room illuminator 310 according to the movement of the support arm 65 and the angle of the reflecting plate 67 with respect to the screen 63. That is, it is possible to turn the illumination of the exterior illuminator 56, the in-bore illuminator 58, and the examination room illuminator 310 into the ON state when the reflecting plate 67 is arranged at the first angle to be substantially horizontal. Accordingly, the patient P in the first positioning mode can visually recognize the gantry housing 51 from the outside of the gantry 11, and thus, it is possible to mitigate the anxiety of the patient P.

In addition, it is possible to turn the illumination of the exterior illuminator 56, the in-bore illuminator 58, and the examination room illuminator 310 into the OFF state or to have a small amount of light when the reflecting plate 67 is arranged at the second angle. Accordingly, it is possible to further clarify and highlight the contrast of the image reflected on the reflecting plate 67 and to provide environment inside the examination space in which it is possible to allow the patient P to visually recognize the cleaner video according to the magnetic resonance diagnostic apparatus 10 according to the fifth application example.

In the above-described manner, it is possible to provide the environment inside the examination space in which the anxiety of the patient P is improved without causing the uncomfortable feeling to the patient P and the patient P can relax by controlling each lighting pattern of various illuminators based on the output from the detector 72 according to the magnetic resonance diagnostic apparatus 10 according to the fifth application example.

(Sixth Application Example)

A sixth application example corresponds to a case in which the light emitter is mounted to at least one exterior surface of the support arm 65 and the reflecting plate 67. At this time, the light emitter (not illustrated) is controlled by the illumination state changing function 384 in the system control circuitry 38. The light emitter includes at least one light-emitting diode. Incidentally, the light emitter may include a plurality of light-emitting diodes which generate light with a plurality of hues, respectively. In addition, a light source to be mounted to the light emitter is not limited to the light-emitting diode, and may employ an arbitrary light-emitting device.

At least one light emitter is mounted to at least one exterior surface of the support arm 65 and the reflecting plate. For example, the light emitter is mounted to a side face of the support arm 65 on the inner wall 57 side which is on the lateral side. In addition, the light emitter is mounted on the exterior of the base cover 675 in the reflecting plate 67, for example, on the inner wall 57 side which is on the upper side. Incidentally, the illumination state changing function 384 according to the present application example can be executed along with the illumination state changing function 384 according to another application example and at least one function of other various state changing functions.

(Illumination State Changing Function)

The system control circuitry 38 that realizes the illumination state changing function 384 switches the ON state and the OFF state of the illumination or the intensity of the illumination of the light emitter according to at least one of the angle of the reflecting plate 67 with respect to the screen 63 and the relative position of the support arm 65 with respect to the screen 63. The switching between the ON state and the OFF state of the illumination corresponds to, for example, the supply and cut-off of current (switching of current). In addition, the switching of intensity of the illumination corresponds to, for example, the magnitude of the supply amount of current or the change of the number of LED's to which current is supplied.

To be specific, the system control circuitry 38 switches the ON state and the OFF state of the illumination or the intensity of the illumination of the light emitter based on the detection result from the detector 72. The switching between the ON state and the OFF state of the illumination corresponds to, for example, the supply and cut-off of current (switching of current). In addition, the switching of intensity of the illumination corresponds to, for example, the magnitude of the supply amount of current or a change of the number of LED's to which current is supplied. The control of the light emitter relating to the illumination state changing function 384 is similar to that in the above-described fifth application example, and thus, will not be described.

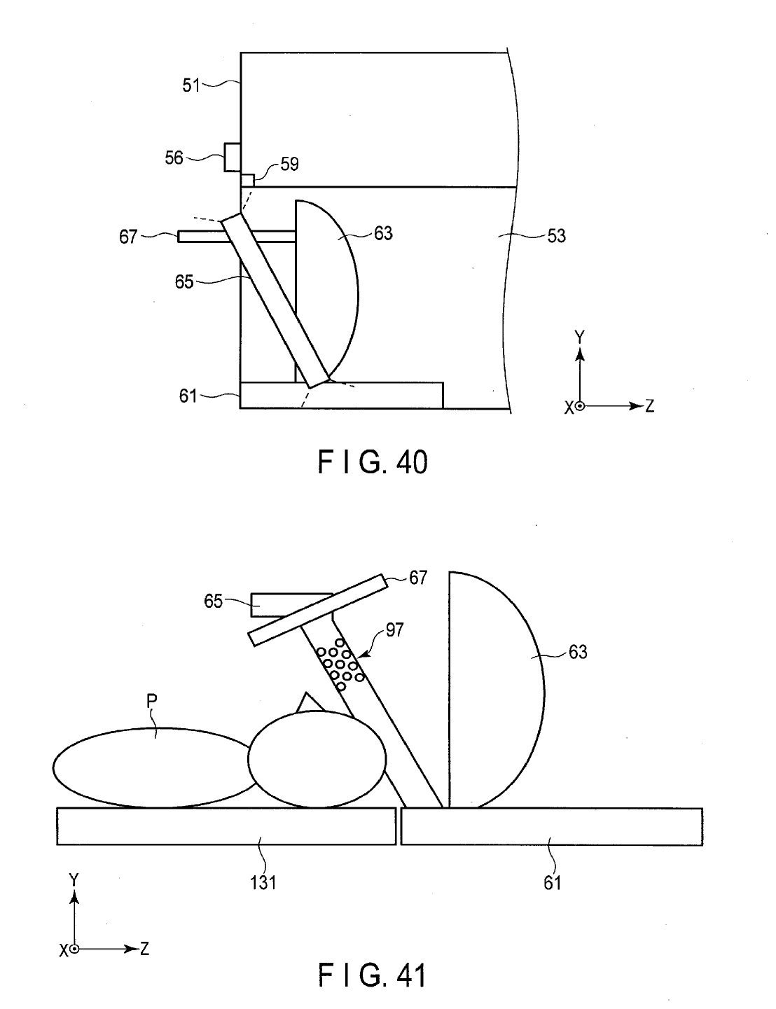

FIG. 40 is a diagram illustrating an example of an illumination state of the light emitter when the reflecting plate 67 is arranged at the first angle as a positioning state in the first positioning mode. At this time, the illumination of the light emitter is in the ON state as illustrated in FIG. 40.

According to the above-described configuration, it is possible to obtain effects to be described as follows.

According to the magnetic resonance diagnostic apparatus 10 according to the sixth application example, it is possible to switch a lighting pattern (ON state and the OFF state or the intensity of the illumination) of illumination of the light emitter, which is mounted to at least one exterior surface of the support arm 65 and the reflecting plate 67, according to the movement of the support arm 65 and the angle of the reflecting plate 67 with respect to the screen 63. That is, it is possible to turn the illumination of the light emitter into the ON state when the reflecting plate 67 is arranged at the first angle to be substantially horizontal. Accordingly, it is possible to decrease a shadow at the end on the couch side of the bore 53 which is caused by the reflecting plate 67 and the support arm 65 in the first positioning mode.

In the above-described manner, when each lighting pattern of various illuminators is controlled based on the output from the detector 72, it is possible to make the reflecting plate 67 and the support arm 65 to be almost optically synchronized (integrated) with the projected light LP passing through the image of the screen 63 or the gap G1 and to reduce a sense of existence of the reflecting plate 67 and the support arm 65 in the first positioning mode according to the magnetic resonance diagnostic apparatus 10 according to the sixth application example. Accordingly, it is possible to provide the environment of the examination room in which the uncomfortable feeling near the bore 53 that the patient P in the first positioning mode feels is reduced, the anxiety of the patient P is improved, and the patient P can relax.

In addition, it is possible to turn the illumination of the light emitter into the OFF state when the reflecting plate 67 is arranged at the second angle. Accordingly, it is possible to provide the environment inside the examination space in which it is possible to clarify (highlight) the contrast of the image reflected on the reflecting plate 67. In this manner, the patient P can visually recognize the contrast of the image reflected on the reflecting plate 67 more clearly.

(Seventh Application Example)

A seventh application example corresponds to a case in which a speaker is mounted to at least one exterior surface of the support arm 65 and the reflecting plate 67. The speaker is controlled by the system control circuitry 38 using an acoustic state changing function to be described later in the seventh application example.

FIG. 41 is a side view of the movable screen apparatus 15 according to the seventh application example. As illustrated in FIG. 41, a speaker 97 is provided in the movable screen apparatus 15 according to the seventh application example. To be specific, the speaker 97 is preferably provided on the exterior surface of the support arm 65 which is arranged at a position close to the head of the patient P. The speaker 97 generates sounds depending on various uses.

Incidentally, the speaker 97 may be provided on at least one exterior surface of the support arm 65 and the reflecting plate 67. For example, the speaker 97 is provided on a side face of the base cover 675 in the reflecting plate 67. The speaker 97 is configured using a non-magnetic material. Incidentally, the speaker 97 may have a magnetically shielded property.

The speaker 97 is connected to the projector control apparatus 200 in a wired or wireless manner. In this case, the speaker 97 may generate a sound that corresponds to the image projected from the projector 100 to the movable screen apparatus 15 and is transmitted from the projector control apparatus 200, for example. The patient P can view the image while listening to the sound, and thus, it is possible to further comfortably stay inside the bore 53.

In addition, the speaker 97 may be connected to the console 27 in a wired or wireless manner. In this case, the speaker 97 may generate a sound of a medical professional which has been collected through a microphone provided in the console 27. Accordingly, it is possible to deliver an instruction or the like of the medical professional to the patient P during the MR imaging.

(Acoustic State Changing Function)

An acoustic state changing function 385 is a function of changing an acoustic state of a space (inside the bore 53) in which the movable screen apparatus 15 is arranged according to the angle of the reflecting plate 67 with respect to the screen 63 and the relative position of the support arm 65 with respect to the screen 63, or the attachment and detachment of the reflecting plate 67 to and from the support arm 65. Hereinafter, a process according to the acoustic state changing function 385 will be described. Incidentally, the acoustic state changing function 385 can be executed along with at least one function of other various state changing functions.

To be specific, the system control circuitry 38 that realizes the acoustic state changing function 385 changes sound characteristics generated by the speaker 97 based on the detection result from the detector 72 in order to change the acoustic state. The acoustic state of the space (inside the bore 53) in which the movable screen apparatus 15 is arranged changes depending on the change of the sound characteristics. Examples of the sound characteristics include volume (sound pressure), tone (waveform), pitch (frequency), reproduction speed of sound, and the like.

Incidentally, the system control circuitry 38 may switch the sound characteristics and a sound source in response to switching of the image projected on the screen. In addition, the system control circuitry 38 may change a music output from the speaker 97 based on the detection result from the detector 72.

Sound characteristics (hereinafter, referred to as first acoustic characteristics) output from the speaker 97 when the first angle is detected by the detector 72 or when the support arm 65 is positioned on the screen 63 side on the movable carriage 61 are different from sound characteristics (hereinafter, referred to as second acoustic characteristics) output from the speaker 97 when the second angle is detected by the detector 72 or when the support arm 65 is positioned on the couch 13 side on the movable carriage 61.

For example, the volume of the first acoustic characteristics is larger than the volume of the second acoustic characteristics. Each difference between the tone, the pitch, and the reproduction speed of the first acoustic characteristics and the tone, the pitch, and the reproduction speed of the second acoustic characteristics depends on a difference between surroundings of the patient P in the first positioning mode, for example, environment (hereinafter, referred to as a first sound field environment) of a sound field in the examination room 300 and surroundings of the patient P in the second positioning mode, for example, environment (hereinafter, referred to as a second sound field environment) of a sound field inside the bore 53.

The first sound field environment and the second sound field environment are different from each other in terms of dispersion, reflection, absorption and the like of sound. In addition, the first sound field environment and the second sound field environment are different from each other in terms of characteristics of acoustic problems such as booming and flutter echo. Incidentally, the first acoustic characteristics and the second acoustic characteristics may be optimized according to each sound field environment, and stored in the main memory circuitry 37.

The system control circuitry 38 causes a sound having the first acoustic characteristics to be output from the speaker 97 when the first angle is detected by the detector 72 or when the support arm 65 is positioned on the screen 63 side on the movable carriage 61. A period in which the first acoustic characteristics are maintained corresponds to, for example, the period from Step Sa1 to No in Step Sa4 and the period from Step Sa8 to the end of the flowchart in FIG. 29.

The system control circuitry 38 causes a sound having the second acoustic characteristics to be output from the speaker 97 when the second angle is detected by the detector 72 or when the support arm 65 is positioned on the couch 13 side on the movable carriage 61. A period in which the second acoustic characteristics are maintained corresponds to, for example, the period from Yes in Step Sa4 to Step Sa7 of the flowchart in FIG. 29, for example.

When the positioning state of the reflecting plate 67 is changed from the first angle to the second angle, that is, when the start time of the rotating operation of the reflecting plate 67 about the rotation shaft RR1 is detected, the system control circuitry 38 changes the sound characteristics, output from the speaker 97, from the first acoustic characteristics to the second acoustic characteristics. The process of changing the sound characteristics from the first acoustic characteristics to the second acoustic characteristics corresponds to, for example, Step Sa5 of the flowchart in FIG. 29.

When the positioning state of the reflecting plate 67 is changed from the second angle to the first angle, the system control circuitry 38 changes the sound characteristics, output from the speaker 97, from the second acoustic characteristics to the first acoustic characteristics. The process of changing the sound characteristics from the second acoustic characteristics to the first acoustic characteristics corresponds to, for example, Step Sa8 of the flowchart in FIG. 29.

Incidentally, the system control circuitry 38 may change the sound characteristics, output from the speaker 97, from the first acoustic characteristics to the second acoustic characteristics when the support arm 65 is drawn out from the screen 63 side to the couch 13 side on the movable carriage 61 or when the reflecting plate 67 is attached to the support arm 65. In addition, the system control circuitry 38 may change the sound characteristics, output from the speaker 97, from the second acoustic characteristics to the first acoustic characteristics when the support arm 65 is pushed from the couch 13 side to the screen 63 side on the movable carriage 61 or when the reflecting plate 67 is detached from the support arm 65. In addition, the system control circuitry 38 may change the sound characteristics according to the content of the image projected on the screen 63.

According to the above-described configuration, it is possible to obtain effects to be described as follows.

According to the magnetic resonance diagnostic apparatus 10 according to the seventh application example, it is possible to change the acoustic state according to the angle of the reflecting plate 67 with respect to the screen 63 and the relative position of the support arm 65 with respect to the screen 63, or the attachment and detachment of the reflecting plate 67 to and from the support arm 65. That is, it is possible to provide an optimal acoustic state (volume, pitch, tone and the like) to the patient P in response to a distance between the patient P and the speaker 97. Accordingly, it is possible to provide the environment of the examination room in which the uncomfortable feeling of the patient P is reduced, the anxiety of the patient P is improved, and the patient P can relax.

(Eighth Application Example)

An eighth application example corresponds to a case in which the reflecting plate 67 is rotated in response to the changing operation of the positioning state of the support arm 65 or the positioning state of the support arm 65 with respect to the screen 63. That is, the movable screen apparatus 15 in the eighth application example includes a mechanism (hereinafter, referred to as a direction changing mechanism) that changes a direction (angle) of the reflecting plate 67 while being interlocked with the slide of the support arm 65 along the Z-axis.

The direction changing mechanism is configured of a ball screw and a flexible shaft (or various gears, various transmitting mechanisms (chain and sprocket, pulley and belt, a shaft drive and the like), a universal joint), made of, for example, a non-magnetic material. The direction changing mechanism is mounted to the movable carriage 61. The angle of the reflecting plate 67 is changed while being interlocked with the slide of the support arm 65 through the direction changing mechanism.

The support arm 65 includes a non-magnetic gear (hereinafter, referred to as a reflecting plate rotation gear) which is fit to the rotation shaft RR1. At this time, the reflecting plate 67 rotates about the rotation shaft RR1 while being interlocked with rotation of the reflecting plate rotation gear.

A nut included in the ball screw is connected to a sliding member of the sliding mechanism 71, for example, the wheel 651 or a portion of the support arm 65 (a portion of the support arm 65 parallel to the Z direction) which is adjacent to the guide 611, and the like. Accordingly, the nut moves on a threaded shaft of the ball screw along with the slide of the support arm 65. The threaded shaft rotates along with the movement of the nut, that is, the slide of the support arm 65.

An end of the flexible shaft is connected to an end of the threaded shaft outside a movement range of the nut. A gear (hereinafter, referred to as an other-end gear), which is provided at the other end of the flexible shaft, meshes with the reflecting plate rotation gear. The flexible shaft rotates depending on the rotation of the threaded shaft accompanying the slide of the support arm 65. The rotation of the flexible shaft is transferred to the reflecting plate rotation gear through the rotation of the other-end gear.

(Interlocking Rotation Function)

An interlocking rotation function is a function of rotating the reflecting plate 67 about the rotation shaft RR1 while being interlocked with the slide of the support arm 65 along the Z direction on the movable carriage 61. A reflection state of the reflecting plate 67 is changed through the interlocking rotation function. Hereinafter, the interlocking rotation function will be described. Incidentally, the interlocking rotation function can be executed along with at least one function of other various state changing functions.

According to the above-described configuration, the reflecting plate 67 rotates about the rotation shaft RR1 while being interlocked with the slide of the support arm 65. To be specific, the reflecting plate 67 rotates from the first angle to the second angle when the support arm 65 is slid from the screen 63 side to the couch 13 side. In addition, the reflecting plate 67 rotates from the second angle to the first angle when the support arm 65 is slid from the couch 13 side to the screen 63 side.

To be specific, the angle of the reflecting plate 67 is switched from the first angle to the second angle after mode transition from the first positioning mode to the second positioning mode, that is, when the support arm 65 is slid from the screen 63 side to the couch 13 side by the user's operation (when the support arm 65 is drawn out from the movable carriage 61).

In addition, the angle of the reflecting plate 67 is switched from the second angle to first angle after mode transition from the second positioning mode to the first positioning mode, that is, when the support arm 65 is slid from the couch 13 side to the screen 63 side by the user's operation (when the support arm 65 is pushed to the movable carriage 61).

Incidentally, a structure of the direction changing mechanism is not limited to the above-described structure. That is, any structure may be employed as long as the structure enables the slide of the support arm 65 (translational motion) to be converted to the rotating motion of the reflecting plate 67.

According to the magnetic resonance diagnostic apparatus 10 according to the eighth application example, it is possible to rotate the reflecting plate 67 in response to the changing operation of the positioning state of the support arm 65 or the positioning state of the support arm 65 with respect to the screen 63. That is, the magnetic resonance diagnostic apparatus 10 according to the eighth application example can rotate the angle of the reflecting plate 67 to an optimal angle while being interlocked with the slide of the support arm 65 along the Z direction on the movable carriage 61. Accordingly, an operation plac with respect to the user is mitigated, and further, the examination efficiency with respect to the patient P is improved.

Further, the reflecting plate 67 rotates from the first angle to the second angle when the support arm 65 is slid from the screen 63 side to the couch 13 side, and thus, it is possible to reduce the time in which the patient's own face is reflected on the reflecting plate 67. In addition, it is possible to set the time in which the patient's own face is reflected on the reflecting plate 67 to be zero, in fact, by adjusting a gear ratio between various gears in the direction changing mechanism in advance. In the above-described manner, it is possible to provide the environment of the examination room and the examination space in which the uncomfortable feeling of the patient P is reduced, the anxiety of the patient P is improved, and the patient P can relax according to the magnetic resonance diagnostic apparatus 10 according to the eighth application example.

According to at least one embodiment described above, it is possible to improve the gantry the dwelling ability inside the bore. For example, it is possible to acquire (detect) various positional changes in movable parts or detachable parts inside the movable screen apparatus 15 as a trigger signal, and to execute various additional functions the reflection state changing function 382, the photographing state changing function 383, the illumination state changing function 384, the acoustic state changing function 365, and the interlocking rotation function) in the state changing function 381.

That is, for example, it is possible to execute the state changing function 381 (at least one of the reflection state changing function 382, the photographing state changing function 383, the illumination state changing function 384, the acoustic state changing function 365, and the interlocking rotation function) in response to at least one of the positioning state of at least one of the reflecting plate 67 and the support arm 65, and the operation of the positioning state according to the magnetic resonance diagnostic apparatus 10 according to at least one embodiment.

Accordingly, it is possible to improve the effect of the image that is visually recognized by the patient P positioned inside the bore 53. In addition, as described above, another state changing function may be executed in response to detection of the attachment of the reflecting plate 67 to the support arm 65 and detection of the detachment of the reflecting plate 67 from the support arm 65 in the reflection state changing function 382.

(Ninth Application Example)

The present application example corresponds to a case in which an image providing apparatus (image providing apparatus), instead of the movable screen apparatus 15, is arranged immediately above the patient fixing tool 137. The image providing apparatus is supported by a column such as the support arm 65 extending from the couch top 131 or the patient fixing tool 137, for example. In addition, the image providing apparatus may be provided in an upper coil apparatus which is attachable to and detachable from a lower coil apparatus of a head coil apparatus which is placed to the couch top 131. At this time, the attachment and detachment of the image providing apparatus corresponds to the attachment and detachment of the upper coil apparatus from and to the lower coil apparatus. The image providing apparatus provides an image from an image source of the projector 100 for a subject placed on the couch top 131 via a reflecting plate 67 that reflects the image or a display that displays the image. To be specific, the image providing apparatus is realized by the reflecting plate 67, the reflection unit 68, or a predetermined display. The display according to the present application example is, for example, an organic EL display and is formed in a curved shape so as to cover a field-of-view range of the subject P.

The detector 72 is provided in the column, the lower coil apparatus or the like.

In the present application example, various triggers relating to the various functions, which have been described in the above-described embodiment and first to eighth application examples, can be understood by replacing the reflecting plate 67 or the reflection unit 68 of the above-described embodiment and first to eighth application examples suitably to the image providing apparatus, and thus, will not be described. In addition, effects according to the present application example are the same as the effects of the above-described embodiment and first to eighth application examples, and thus, will not be described.

According to the medical image diagnostic apparatus of the above-described embodiment and application examples, it is possible to improve the dwelling ability inside the bore of the gantry.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image diagnostic apparatus comprising:
an image providing apparatus that provides an image from an image source for a subject placed on a couch top via a reflecting plate which reflects the image or a display which displays the image; and
control circuitry that performs at least one of control of illumination provided in an examination room or a gantry and control of a camera which photographs the subject placed on the couch top, according to attachment and detachment of the image providing apparatus or positioning of the image providing apparatus with respect to the couch top.

2. The medical image diagnostic apparatus according to claim 1, wherein
the image providing apparatus is the reflecting plate, and
the apparatus further comprising:
a support arm which rotatably supports the reflecting plate, and
a detector which detects the positioning of the reflecting plate based on at least one of attachment and detachment of the reflecting plate to and from the support arm, a position of the support arm with respect to a screen, and a rotation angle of the reflecting plate.

3. The medical image diagnostic apparatus according to claim 2, wherein
the image source outputs an image to the screen, and
the reflecting plate reflects the image output to the screen.

4. The medical image diagnostic apparatus according to claim 2, wherein
at least one of the camera, which photographs the subject on the couch top, is mounted to at least one of the reflecting plate and the support arm,
the camera includes an objective lens, an optical waveguide which guides light from the objective lens, and a light receiving element which is provided outside a bore and receives the light from the optical waveguide, and
the control circuitry switches a photographing direction of the camera according to at least one of an angle of the reflecting plate with respect to the screen and a relative position of the support arm with respect to the screen.

5. The medical image diagnostic apparatus according to claim 4, further comprising:
an image display monitor that displays an image photographed by the camera,
wherein the control circuitry sets a position, designated by a user, in the image on the image display monitor as a position that needs to be moved to a center position of a magnetic field.

6. The medical image diagnostic apparatus according to claim 2, wherein
at least one of the reflecting plate and the support arm includes a speaker on an exterior surface thereof, and
the control circuitry controls at least one of volume, a waveform, a frequency, and reproduction speed of a sound output from the speaker according to at least one of an angle of the reflecting plate with respect to the screen and a relative position of the support arm with respect to the screen.

7. The medical image diagnostic apparatus according to claim 2, wherein
at least one of the reflecting plate and the support arm includes a light emitter on an exterior surface thereof, and
the control circuitry switches an ON state and an OFF state of light generated by the light emitter, or intensity of the light according to at least one of an angle of the reflecting plate with respect to the screen and a relative position of the support arm with respect to the screen.

8. The medical image diagnostic apparatus according to claim 2, wherein
the image providing apparatus including the support arm rotates the reflecting plate in response to positioning of the support arm with respect to the screen.

9. The medical image diagnostic apparatus according to claim 1, wherein
the illumination is at least one of an in-bore illuminator provided inside a bore formed in the gantry, an exterior illuminator provided on an exterior surface of the gantry, and an examination room illuminator provided in the examination room, and
the control circuitry performs control between an ON state and an OFF state of the illumination or dimming control of the illumination.

10. A medical image diagnostic apparatus comprising:
a reflection unit that includes a beam splitter which has predetermined transmittance and reflectance, a reflecting plate which reflects an image from an image source, and a light source which irradiates the beam splitter with light; and
control circuitry that controls the light source according to positioning of the reflecting plate with respect to the image source.

11. The medical image diagnostic apparatus according to claim 10, wherein
the reflection unit further includes a light guide plate which is arranged on a face of the beam splitter, and
the light source is arranged at an end of the light guide plate.

12. The medical image diagnostic apparatus according to claim 10, wherein
the reflection unit includes a film on which a predetermined image is provided, at a back side of the light guide plate.

13. The medical image diagnostic apparatus according to claim 10 further comprising
a monitor that includes the light source on a face of the beam splitter.

14. A medical image diagnostic apparatus comprising
an image projection apparatus that includes a screen on which an image from a projector is projected, a reflecting plate which reflects the image projected on the screen, and a support arm which rotatably supports the reflecting plate,
wherein the reflecting plate rotates in response to a positioning state of the support arm with respect to the screen or a changing operation of the positioning state.
